(19) United States
(12) Patent Application Publication
(10) Pub. No.: US 2009/0075387 A1
(43) Pub. Date: Mar. 19, 2009

(54) Methods and Compositions for Biomarkers Associated with Change in Physical Performance

(76) Inventors: John E. Kalns, San Antonio, TX (US); Robert J. Christy, San Antonio, TX (US)

Correspondence Address: MYERS BIGEL SIBLEY & SAJOVEC, PO BOX 37428, RALEIGH, NC, 27627, US

(21) Appl. No.: 12/090,194

(22) PCT Filed: Sep. 27, 2006
(86) PCT No.: PCT/US06/37489

§ 371 Date: Oct. 13, 2008

Related U.S. Application Data
(60) Provisional application No. 60/726,792, filed on Oct. 14, 2005.
(60) Provisional application No. 60/808,165, filed on May 24, 2006.

(51) Int. Cl. G01N 33/44; G01N 33/68

(52) U.S. Cl. 436/85; 436/86; 436/87

(57) ABSTRACT

The present invention provides methods and compositions for detecting an improvement in the performance of a physical or athletic activity and/or in a cognitive activity in a subject upon administration to the subject of a performance enhancing material and/or upon contact of the subject with a performance enhancing material and/or upon implementation of a performance enhancing activity by the subject by detecting in the subject a change in a biomarker associated with physical or athletic activity and/or cognitive activity.

### STATEMENT OF PRIORITY

This application claims the benefit, under 35 U.S.C. § 119(e), of U.S. Provisional Application No. 60/726,792, filed Oct. 14, 2005 and U.S. Provisional Application No. 60/808,165, filed May 24, 2006, the entire contents of each of which are incorporated by reference here in their entireties.

### STATEMENT OF GOVERNMENT SUPPORT

This invention was supported through the Army Small Business Technology Transfer (STTR) Program, as topic number A04-T018, proposal number A045-108-0142, awarded to Hyperion Biotechnology, Inc.

### BACKGROUND OF THE INVENTION

The impact of nutritional supplementation and/or training strategies to improve physical performance in mammals can be difficult to assess because measurements of work output or ability to perform a particular task more rapidly are influenced by a multitude of factors that can be difficult to control for. As a result, very large numbers of subjects must be enrolled in studies in order to detect significant differences with acceptable statistical confidence. Evaluating the impact of supplements, drugs, devices, clothing, and/or changes in training regimens on an individual basis can also be difficult, requiring, for example, expensive, bulky apparatus to measure exhaled gases, or periodic evaluation by standardized performance tests.

The present invention provides an improvement over the art by providing methods and compositions for evaluating changes in physical performance in mammals by detecting and/or measuring biomarkers associated with a change in physical performance.

### BRIEF DESCRIPTION OF THE DRAWINGS

FIG. 1. Trial A: carbohydrate; Trial B: placebo. Top panel: biomarker peak height; middle panel: CHO; bottom panel: FAT. During exercise alternating between treadmill and cycle ergometer at intensities varying systematically between 70% to 90% of maximum output, utilization of oxygen (V0₂, liters/minute) and production of CO₂ (VCO₂, liters/minute) was measured. An estimate of the rate of carbohydrate oxidation (CHO, grams/min) was calculated using the formula, CHO=(4.55*VCO₂)−(3.21*VO₂). The rate of fat consumption (FAT, grams/minute) was calculated using, FAT=1.67*(VO₂—VCO₂).

### SUMMARY OF THE INVENTION

The present invention provides a method of detecting an improvement in the performance of a physical or athletic activity in a subject upon administration to the subject of a performance enhancing material and/or upon contact of the subject with a performance enhancing material and/or upon implementation of a performance enhancing activity by the subject, comprising:

a) measuring an amount of a biomarker in a first sample of a test subject prior to performance of the physical or athletic activity;

b) measuring the amount of a biomarker in a first sample of a control subject prior to performance of the physical or athletic activity;

c) administering to the subject the performance enhancing material and/or contacting the subject with the performance enhancing material and/or implementing the performance enhancing activity to the test subject;

d) having the test subject and control subject perform the physical or athletic activity;

e) measuring the amount of the biomarker in a second sample of the test subject and measuring the amount of the biomarker in a second sample of the control subject after step (d);

f) comparing the amount of the biomarker in the first sample and the second sample of the test subject and comparing the amount of the biomarker in the first sample and the second sample of the control subject; and

g) comparing the change in the amount of the biomarker of step (e) in the test subject with the change in the amount of the biomarker of step (e) in the control subject, whereby an increase in the amount of biomarker in the test subject that is greater than an increase in the amount of the biomarker in the control subject detects an improvement in the performance of the physical or athletic activity in the subject upon administration to the subject of the performance enhancing material and/or contacting the subject with the performance enhancing material and/or upon implementation of a performance enhancing activity by the subject.

In addition, the present invention provides a method of detecting a change in the chemical energy source, (e.g., fat and/or carbohydrate) used by the body of a subject during performance of physical or athletic activity, upon administration to the subject of a performance enhancing material and/or upon contact of the subject with a performance enhancing material and/or upon implementation of a performance enhancing activity by the subject, comprising:

a) measuring an amount of a biomarker in a first sample of a test subject prior to performance of the physical or athletic activity;

b) measuring the amount of a biomarker in a first sample of a control subject prior to performance of the physical or athletic activity;

c) administering to the subject the performance enhancing material and/or contacting the subject with the performance enhancing material and/or implementing the performance enhancing activity to the test subject;

d) having the test subject and control subject perform the physical or athletic activity;

e) measuring the amount of the biomarker in a second sample of the test subject and measuring the amount of the biomarker in a second sample of the control subject after step (d);

f) comparing the amount of the biomarker in the first sample and the second sample of the test subject and comparing the amount of the biomarker in the first sample and the second sample of the control subject; and

g) comparing the change in the amount of the biomarker of step (e) in the test subject with the change in the amount of the biomarker of step (e) in the control subject, whereby an increase in the amount of biomarker in the test subject that is greater than or less than an increase in the amount of the biomarker in the control subject detects a change in the chemical energy source, (e.g., fat and/or carbohydrate) used by the body of the subject during performance of physical or athletic activity, upon administration to the subject of a performance enhancing material and/or upon contact of the subject with a performance enhancing material and/or upon implementation of a performance enhancing activity by the subject.

Further provided herein is a method for measuring an improvement in the ability of a subject to perform a cognitive activity upon administration to the subject of a performance enhancing material and/or upon contact of the subject with a performance enhancing material and/or upon implementation of a performance enhancing activity by the subject, comprising:

a) measuring an amount of a biomarker in a first sample of a test subject prior to performance of the cognitive activity;

b) measuring the amount of a biomarker in a first sample of a control subject prior to performance of the cognitive activity;

c) administering to the subject the performance enhancing material and/or contacting the subject with the performance enhancing material and/or implementing the performance enhancing activity to the test subject;

d) having the test subject and control subject perform the cognitive activity;

e) measuring the amount of the biomarker in a second sample of the test subject and measuring the amount of the biomarker in a second sample of the control subject after step (d);

f) comparing the amount of the biomarker in the first sample and the second sample of the test subject and comparing the amount of the biomarker in the first sample and the second sample of the control subject; and

g) comparing the change in the amount of the biomarker of step (e) in the test subject with the change in the amount of the biomarker of step (e) in the control subject, whereby an increase in the amount of biomarker in the test subject that is greater than an increase in the amount of the biomarker in the control subject detects an improvement in the ability of the test subject to perform a cognitive activity upon administration to the subject of a performance enhancing material and/or upon contact of the subject with a performance enhancing material and/or upon implementation of a performance enhancing activity by the subject.

The present invention also provides a method of detecting a decrement in the performance of a physical or athletic activity in a subject upon administration to the subject of a performance enhancing material and/or upon contact of the subject with a performance enhancing material and/or upon implementation of a performance enhancing activity by the subject, comprising:

a) measuring an amount of a biomarker in a first sample of a test subject prior to performance of the physical or athletic activity;

b) measuring the amount of a biomarker in a first sample of a control subject prior to performance of the physical or athletic activity;

c) administering to the subject the performance enhancing material and/or contacting the subject with the performance enhancing material and/or implementing the performance enhancing activity to the test subject;

d) having the test subject and control subject perform the physical or athletic activity;

e) measuring the amount of the biomarker in a second sample of the test subject and measuring the amount of the biomarker in a second sample of the control subject after step (d);

f) comparing the amount of the biomarker in the first sample and the second sample of the test subject and comparing the amount of the biomarker in the first sample and the second sample of the control subject; and

g) comparing the change in the amount of the biomarker of step (e) in the test subject with the change in the amount of the biomarker of step (e) in the control subject, whereby an increase in the amount of biomarker in the test subject that is less than an increase in the amount of the biomarker in the control subject detects a decrement in the performance of the physical or athletic activity in the subject upon administration to the subject of the performance enhancing material and/or contacting the subject with the performance enhancing material and/or upon implementation of a performance enhancing activity by the subject.

In addition, the present invention provides a method for measuring a decrement in the ability of a subject to perform a cognitive activity upon administration to the subject of a performance enhancing material and/or upon contact of the subject with a performance enhancing material and/or upon implementation of a performance enhancing activity by the subject, comprising:

a) measuring an amount of a biomarker in a first sample of a test subject prior to performance of the cognitive activity;

b) measuring the amount of a biomarker in a first sample of a control subject prior to performance of the cognitive activity;

c) administering to the subject the performance enhancing material and/or contacting the subject with the performance enhancing material and/or implementing the performance enhancing activity to the test subject;

d) having the test subject and control subject perform the cognitive activity;

e) measuring the amount of the biomarker in a second sample of the test subject and measuring the amount of the biomarker in a second sample of the control subject after step (d);

f) comparing the amount of the biomarker in the first sample and the second sample of the test subject and comparing the amount of the biomarker in the first sample and the second sample of the control subject; and

g) comparing the change in the amount of the biomarker of step (e) in the test subject with the change in the amount of the biomarker of step (e) in the control subject, whereby an increase in the amount of biomarker in the test subject that is less than an increase in the amount of the biomarker in the control subject detects a decrement in the ability of the test subject to perform a cognitive activity upon administration to the subject of a performance enhancing material and/or upon contact of the subject with a performance enhancing material and/or upon implementation of a performance enhancing activity by the subject.

Further provided herein is a method for identifying a material and/or activity that enhances the performance of a physical or athletic activity and/or the cognitive performance by a subject, comprising:

a) measuring an amount of a biomarker in a first sample from a test subject prior to performance of the physical or athletic activity and/or cognitive activity;

b) having the test subject perform the physical or athletic activity and/or cognitive activity in the absence of the potentially performance enhancing and/or cognitive performance enhancing material;

c) measuring an amount of the biomarker in a second sample from the test subject after step (b);

d) administering to the subject the potentially performance enhancing and/or cognitive performance enhancing material and/or contacting the subject with the potentially performance enhancing and/or cognitive enhancing material and/or having the subject implement the potentially performance enhancing and/or cognitive enhancing activity;

e) having the test subject perform the physical or athletic activity and or cognitive activity after completion of step (d);

f) measuring the amount of the biomarker in a third sample from the test subject after step (e);

g) comparing the amount of the biomarker in the first sample and the second sample of the test subject;

h) comparing the amount of the biomarker in the first sample and the third sample of the test subject; and

i) comparing the change in the amount of the biomarker of step (g) with the change in the amount of the biomarker of step (h) whereby an increase in the amount of biomarker of step (h) that is greater than an increase in the amount of the biomarker of step (g) identifies a material that enhances the performance of a physical or athletic activity and/or cognitive activity by a subject.

The present invention additionally provides a method for identifying an improvement in the performance of a physical or athletic activity by a subject over time upon administration to the subject of a performance enhancing material and/or upon contact of the subject with a performance enhancing material and/or upon implementation of a performance enhancing activity by the subject, comprising:

a) measuring an amount of a biomarker in a first sample of a test subject prior to performance of the physical or athletic activity;

b) administering to the subject the performance enhancing material and/or contacting the subject with the performance enhancing material and/or implementing the performance enhancing activity to the test subject at a first time point;

c) having the test subject perform the physical or athletic activity at a first time point;

d) measuring the amount of the biomarker in a second sample of the test subject after step (c);

e) administering to the subject the performance enhancing material and/or contacting the subject with the performance enhancing material and/or implementing the performance enhancing activity to the test subject at a second time point;

f) having the test subject perform the physical or athletic activity at a second time point;

g) measuring the amount of the biomarker in a third sample of the test subject after step (f);

h) comparing the amount of the biomarker in the first sample and the second sample of the test subject;

i) comparing the amount of the biomarker in the first sample and the third sample of the test subject; and

j) comparing the change in the amount of the biomarker of step (h) in the test subject with the change in the amount of the biomarker of step (i) in the test subject, whereby an increase in the amount of biomarker of step (i) that is greater than an increase in the amount of the biomarker of step (h) identifies an improvement in the performance of a physical or athletic activity in the subject over time upon administration to the subject of a performance enhancing material and/or upon contact of the subject with a performance enhancing material and/or upon implementation of a performance enhancing activity by the subject.

Further provided herein is a method for identifying an improvement in the performance of a cognitive activity in a subject over time upon administration to the subject of a performance enhancing material and/or upon contact of the subject with a performance enhancing material and/or upon implementation of a performance enhancing activity by the subject, comprising:

a) measuring an amount of a biomarker in a first sample of a test subject prior to performance of the cognitive activity;

b) administering to the subject the performance enhancing material and/or contacting the subject with the performance enhancing material and/or implementing the performance enhancing activity to the test subject at a first time point;

c) having the test subject perform the cognitive activity at a first time point;

d) measuring the amount of the biomarker in a second sample of the test subject after step (c);

e) administering to the subject the performance enhancing material and/or contacting the subject with the performance enhancing material and/or implementing the performance enhancing activity to the test subject at a second time point;

f) having the test subject perform the cognitive activity at a second time point;

g) measuring the amount of the biomarker in a third sample of the test subject after step (f);

h) comparing the amount of the biomarker in the first sample and the second sample of the test subject;

i) comparing the amount of the biomarker in the first sample and the third sample of the test subject; and

j) comparing the change in the amount of the biomarker of step (h) in the test subject with the change in the amount of the biomarker of step (i) in the test subject, whereby an increase in the amount of biomarker of step (i) that is greater than an increase in the amount of the biomarker of step (h) identifies an improvement in the performance of a cognitive activity in the subject over time upon administration to the subject of a performance enhancing material and/or upon contact of the subject with a performance enhancing material and/or upon implementation of a performance enhancing activity by the subject.

Also provided herein is a method for identifying an improvement in the performance of a physical or athletic activity by a subject upon administration to the subject of a performance enhancing material and/or upon contact of the subject with a performance enhancing material and/or upon implementation of a performance enhancing activity by the subject, comprising:

a) measuring an amount of a biomarker in a first sample of a test subject prior to performance of the physical or athletic activity;

b) having the test subject perform the physical or athletic activity;

c) measuring the amount of the biomarker in a second sample of the test subject of step (b);

d) measuring an amount of the biomarker in a third sample of the test subject prior to performance of the physical activity;

e) administering to the subject the performance enhancing material and/or contacting the subject with the performance enhancing material and/or having the test subject implement the performance enhancing activity;

f) having the test subject perform the same physical or athletic activity as in step (b);

g) measuring the amount of the biomarker in a fourth sample of the test subject after step (f);

h) comparing the amount of the biomarker in the first sample and the second sample of the test subject;

i) comparing the amount of the biomarker in the third sample and the fourth sample of the test subject; and

j) comparing the change in the amount of the biomarker of step (h) in the test subject with the change in the amount of the biomarker of step (i) in the test subject, whereby an increase in the amount of biomarker of step (i) that is greater than an increase in the amount of the biomarker of step (h) identifies an improvement in the performance of a physical or athletic activity in the subject upon administration to the subject of a performance enhancing material and/or upon contact of the subject with a performance enhancing material and/or upon implementation of a performance enhancing activity by the subject.

Furthermore, the present invention provides a method for identifying an improvement in the performance of a cognitive activity by a subject upon administration to the subject of a performance enhancing material and/or upon contact of the subject with a performance enhancing material and/or upon implementation of a performance enhancing activity by the subject, comprising:

a) measuring an amount of a biomarker in a first sample of a test subject prior to performance of the cognitive activity;

b) having the test subject perform the cognitive activity;

c) measuring the amount of the biomarker in a second sample of the test subject of step (b);

d) measuring an amount of the biomarker in a third sample of the test subject prior to performance of the cognitive activity;

e) administering to the subject the performance enhancing material and/or contacting the subject with the performance enhancing material and/or having the test subject implement the performance enhancing activity;

f) having the test subject perform the same cognitive activity as in step (b);

g) measuring the amount of the biomarker in a fourth sample of the test subject after step (f);

h) comparing the amount of the biomarker in the first sample and the second sample of the test subject;

i) comparing the amount of the biomarker in the third sample and the fourth sample of the test subject; and

j) comparing the change in the amount of the biomarker of step (h) in the test subject with the change in the amount of the biomarker of step (i) in the test subject, whereby an increase in the amount of biomarker of step (i) that is greater than an increase in the amount of the biomarker of step (h) identifies an improvement in the performance of a physical or athletic activity in the subject upon administration to the subject of a performance enhancing material and/or upon contact of the subject with a performance enhancing material and/or upon implementation of a performance enhancing activity by the subject.

Additionally provided is a method of correlating an increase in the amount of a biomarker in a subject with an improvement in the performance of a physical or athletic activity by the subject upon administration to the subject of a performance enhancing material and/or upon contact of the subject with a performance enhancing material and/or upon implementation of a performance enhancing activity by the subject, comprising:

a) detecting in the subject an improvement in the performance of a physical or athletic activity upon administration to the subject of a performance enhancing material and/or upon contact of the subject with a performance enhancing material and/or upon implementation of a performance enhancing activity by the subject by measuring exhaled gases; a change in body composition, including percentage body fat, or percentage muscle; a maximum work output as measured on standard tests including cycle ergometers, treadmills or other machines where energy expenditure over a period of time or maximum output can be measured; and/or a reduction in the amount of time required to perform a standardized physical task including the time required to run, hike, bike, swim or walk a known distance, etc.;

b) detecting in the subject of (a) an increase in the amount of a biomarker in the subject; and

c) correlating the increase in the amount of the biomarker of step (b) with an improvement in the performance of a physical or athletic activity in the subject upon administration to the subject of a performance enhancing material and/or upon contact of the subject with a performance enhancing material and/or upon implementation of a performance enhancing activity by the subject.

In additional embodiments, the present invention provides a method of correlating an increase in the amount of a biomarker in a subject with an improvement in the performance of a cognitive activity by the subject upon administration to the subject of a performance enhancing material and/or upon contact of the subject with a performance enhancing material and/or upon implementation of a performance enhancing activity by the subject, comprising:

a) detecting in the subject an improvement in the performance of a cognitive activity upon administration to the subject of a performance enhancing material and/or upon contact of the subject with a performance enhancing material and/or upon implementation of a performance enhancing activity by the subject by measuring cognitive activity upon administration of, for example, Folstein's Mini-Mental State Examination (MMSE), Peabody Picture Vocabulary Test, Peabody Developmental Motor Scale, Stanford-Binet Intelligence Scale, Battelle Developmental Inventory, Vineland Adaptive Behavior Scale, Woodcock-Johnson Psycho-Educational Battery-Revised, Standard and Supplemental Batteries (WJPEB-R), Wechsler Adult Intelligence Scale—Revised or III (WAIS-R or WAIS III), Wechsler Intelligence Scale for Children (WISC-R or WISC-III), Woodcock-Johnson Psycho-Educational Battery, Wechsler Individual Achievement Test (WIAT), Stanford Test of Academic Skills (STAS), Scholastic Abilities Test for Adults (SATA), Detroit tests of Learning Aptitude-Adult 2, 3, Stanford Diagnostic Mathematics Test, Formal Reading Inventory, Woodcock Reading Mastery Tests-Revised, Gray Oral Reading Test, LSAT, SAT, GMAT, GRE and/or computerized test batteries that measure response time, ability to perform standardized calculations, tasks to measure response time to a visual cue, scores on various computer games and other tasks, on a computerized instrument including handheld devices, PDAs, laptops and desktop computers;

b) detecting in the subject of (a) an increase in the amount of a biomarker in the subject; and

c) correlating the increase in the amount of the biomarker of step (b) with an improvement in the performance of a cognitive activity in the subject upon administration to the subject of a performance enhancing material and/or upon contact of the subject with a performance enhancing material and/or upon implementation of a performance enhancing activity by the subject.

Also provided herein is a method of correlating a decrease in the amount of a biomarker in a subject with a decrement in the performance of a physical or athletic activity by the subject upon administration to the subject of a performance enhancing material and/or upon contact of the subject with a performance enhancing material and/or upon implementation of a performance enhancing activity by the subject, comprising:

a) detecting in the subject a decrement in the performance of a physical or athletic activity upon administration to the subject of a performance enhancing material and/or upon contact of the subject with a performance enhancing material and/or upon implementation of a performance enhancing activity by the subject by measuring exhaled gases; change in body composition, including percentage body fat, or percentage muscle; a maximum work output as measured on standard tests including cycle ergometers, treadmills or other machines where energy expenditure over a period of time or maximum output can be measured, and/or an increase in the amount of time required to perform a standardized physical task including the time required to run, hike, bike, swim or walk a known distance, etc.;

b) detecting in the subject of (a) a decrease in the amount of a biomarker in the subject; and

c) correlating the decrease in the amount of the biomarker of step (b) with a decrement in the performance of a physical or athletic activity in the subject upon administration to the subject of a performance enhancing material and/or upon contact of the subject with a performance enhancing material and/or upon implementation of a performance enhancing activity by the subject.

Further provided herein is a method of correlating a decrease in the amount of a biomarker in a subject with a decrement in the performance of a cognitive activity by the subject upon administration to the subject of a performance enhancing material and/or upon contact of the subject with a performance enhancing material and/or upon implementation of a performance enhancing activity by the subject, comprising:

a) detecting in the subject a decrement in the performance of a cognitive activity upon administration to the subject of a performance enhancing material and/or upon contact of the subject with a performance enhancing material and/or upon implementation of a performance enhancing activity by the subject by measuring cognitive activity upon administration of, for example, Folstein's Mini-Mental State Examination (MMSE), Peabody Picture Vocabulary Test, Peabody Developmental Motor Scale, Stanford-Binet Intelligence Scale, Battelle Developmental Inventory, Vineland Adaptive Behavior Scale, Woodcock-Johnson Psycho-Educational Battery-Revised, Standard and Supplemental Batteries (WJPEB-R), Wechsler Adult Intelligence Scale—Revised or III (WAIS-R or WAIS III), Wechsler Intelligence Scale for Children (WISC-R or WISC-III), Woodcock-Johnson Psycho-Educational Battery, Wechsler Individual Achievement Test (WIAT), Stanford Test of Academic Skills (STAS), Scholastic Abilities Test for Adults (SATA), Detroit tests of Learning Aptitude-Adult 2, 3, Stanford Diagnostic Mathematics Test, Formal Reading Inventory, Woodcock Reading Mastery Tests-Revised, Gray Oral Reading Test, LSAT, SAT, GMAT, GRE and/or computerized test batteries that measure response time, ability to perform standardized calculations, tasks to measure response time to a visual cue, scores on various computer games and other tasks, on a computerized instrument including handheld devices, PDAs, laptops and desktop computers;

b) detecting in the subject of (a) a decrease in the amount of a biomarker in the subject; and

c) correlating the decrease in the amount of the biomarker of step (b) with a decrement in the performance of a cognitive activity in the subject upon administration to the subject of a performance enhancing material and/or upon contact of the subject with a performance enhancing material and/or upon implementation of a performance enhancing activity by the subject.

In yet further embodiments, the present invention provides a method of identifying an improvement in the performance of a physical or athletic activity in a subject upon administration to the subject of a performance enhancing material and/or upon contact of the subject with a performance enhancing material and/or upon implementation of a performance enhancing activity by the subject, comprising:

a) correlating an increase in the amount of a biomarker in the subject with an improvement in the performance of a physical or athletic activity in the subject upon administration to the subject of a performance enhancing material and/or upon contact of the subject with a performance enhancing material and/or upon implementation of a performance enhancing activity by the subject; and

b) detecting an increase in the biomarker of step (a) in the subject, thereby identifying an improvement in the performance of a physical or athletic activity in the subject upon administration to the subject of a performance enhancing material and/or upon contact of the subject with a performance enhancing material and/or upon implementation of a performance enhancing activity by the subject

Additionally provided herein is a method of identifying an improvement in the performance of a cognitive activity in a subject upon administration to the subject of a performance enhancing material and/or upon contact of the subject with a performance enhancing material and/or upon implementation of a performance enhancing activity by the subject, comprising:

a) correlating an increase in the amount of a biomarker in the subject with an improvement in the performance of a cognitive activity in the subject upon administration to the subject of a performance enhancing material and/or upon contact of the subject with a performance enhancing material and/or upon implementation of a performance enhancing activity by the subject; and

b) detecting an increase in the biomarker of step (a) in the subject, thereby identifying an improvement in the performance of a cognitive activity in the subject upon administration to the subject of a performance enhancing material and/or upon contact of the subject with a performance enhancing material and/or upon implementation of a performance enhancing activity by the subject.

Further provided herein is a method of identifying a decrement in the performance of a physical or athletic activity in a subject upon administration to the subject of a performance enhancing material and/or upon contact of the subject with a performance enhancing material and/or upon implementation of a performance enhancing activity by the subject, comprising:

a) correlating a decrease in the amount of a biomarker in the subject with a decrement in the performance of a physical or athletic activity in the subject upon administration to the subject of a performance enhancing material and/or upon contact of the subject with a performance enhancing material and/or upon implementation of a performance enhancing activity by the subject; and

b) detecting a decrease in the biomarker of step (a) in the subject, thereby identifying a decrement in the performance of a physical or athletic activity in the subject upon administration to the subject of a performance enhancing material and/or upon contact of the subject with a performance enhancing material and/or upon implementation of a performance enhancing activity by the subject.

The present invention additionally provides a method of identifying a decrement in the performance of a cognitive activity in a subject upon administration to the subject of a performance enhancing material and/or upon contact of the subject with a performance enhancing material and/or upon implementation of a performance enhancing activity by the subject, comprising:

a) correlating a decrease in the amount of a biomarker in the subject with a decrement in the performance of a cognitive activity in the subject upon administration to the subject of a performance enhancing material and/or upon contact of the subject with a performance enhancing material and/or upon implementation of a performance enhancing activity by the subject; and

b) detecting a decrease in the biomarker of step (a) in the subject, thereby identifying a decrement in the performance of a physical or athletic activity in the subject upon administration to the subject of a performance enhancing material and/or upon contact of the subject with a performance enhancing material and/or upon implementation of a performance enhancing activity by the subject.

### DETAILED DESCRIPTION OF THE INVENTION

As used herein, “a” or “an” or “the” can mean one or more than one. For example, “a” cell can mean one cell or a plurality of cells.

Also as used herein, “and/or” refers to and encompasses any and/or all possible combinations of one or more of the associated listed items, as well as the lack of and/or combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations and variations thereof.

The present invention is based on the unexpected discovery of biomarkers associated with physical or athletic activity and/or cognitive activity in a subject and the ability to identify changes in the performance of a physical or athletic activity and/or cognitive activity by a subject upon administration to the subject of a performance enhancing material and/or upon contact of the subject with a performance enhancing material and/or upon implementation of a performance enhancing activity by the subject by detecting, measuring and/or monitoring these biomarkers.

As used herein, “biomarker” can mean any chemical or biological entity that is produced by cells and/or by commensal flora, or substances that are produced by cells or commensal flora that are then chemically modified by extracellular enzymes, free radicals produced by cells of the body, and/or other naturally occurring processes that are found, for example, in the saliva, urine, blood, vaginal secretion, tears, feces, sputum, hair, nails, skin, wound fluid, nasal swab, lymph, perspiration, swab of oral mucosa, vaginal mucosa, or the anus, or in serum or plasma obtained from blood.

In some embodiments, a biomarker of this invention can be, but is not limited to, a peptide or polypeptide comprising, consisting essentially of and/or consisting of the amino acid sequence of any of SEQ ID NOs:1-36 provided herein. Other examples of biomarkers of this invention are provided in Tables 4, 5 and 6 provided herein and described in the Examples. Thus, in certain embodiments, the present invention is directed to a peptide or polypeptide as described herein and the present invention can employ or involve one or more of the peptides and polypeptides set forth herein in any method and/or kit of this invention, singly and/or in any combination.

Thus, the present invention provides a method of detecting an improvement in the performance of a physical or athletic activity in a subject upon administration to the subject of a performance enhancing material and/or upon contact of the subject with a performance enhancing material and/or upon implementation of a performance enhancing activity by the subject, comprising: a) measuring an amount of a biomarker in a first sample of a test subject prior to performance of the physical or athletic activity; b) measuring the amount of a biomarker in a first sample of a control subject prior to performance of the physical or athletic activity; c) administering to the subject the performance enhancing material and/or contacting the subject with the performance enhancing material and/or implementing the performance enhancing activity to the test subject; d) having the test subject and control subject perform the physical or athletic activity; e) measuring the amount of the biomarker in a second sample of the test subject and measuring the amount of the biomarker in a second sample of the control subject after step (d); f) comparing the amount of the biomarker in the first sample and the second sample of the test subject and comparing the amount of the biomarker in the first sample and the second sample of the control subject; and g) comparing the change in the amount of the biomarker of step (e) in the test subject with the change in the amount of the biomarker of step (e) in the control subject, whereby an increase in the amount of biomarker in the test subject that is greater than an increase in the amount of the biomarker in the control subject detects an improvement in the performance of the physical or athletic activity in the subject upon administration to the subject of the performance enhancing material and/or contacting the subject with the performance enhancing material and/or upon implementation of a performance enhancing activity by the subject.

Further provided herein is a method of detecting a change in the chemical energy source, (e.g., fat and/or carbohydrate) used by the body of a subject during performance of physical or athletic activity, upon administration to the subject of a performance enhancing material and/or upon contact of the subject with a performance enhancing material and/or upon implementation of a performance enhancing activity by the subject, comprising: a) measuring an amount of a biomarker in a first sample of a test subject prior to performance of the physical or athletic activity; b) measuring the amount of a biomarker in a first sample of a control subject prior to performance of the physical or athletic activity; c) administering to the subject the performance enhancing material and/or contacting the subject with the performance enhancing material and/or implementing the performance enhancing activity to the test subject; d) having the test subject and control subject perform the physical or athletic activity; e) measuring the amount of the biomarker in a second sample of the test subject and measuring the amount of the biomarker in a second sample of the control subject after step (d); f) comparing the amount of the biomarker in the first sample and the second sample of the test subject and comparing the amount of the biomarker in the first sample and the second sample of the control subject; and g) comparing the change in the amount of the biomarker of step (e) in the test subject with the change in the amount of the biomarker of step (e) in the control subject, whereby an increase in the amount of biomarker in the test subject that is greater than or less than an increase in the amount of the biomarker in the control subject detects a change in the chemical energy source, (e.g., fat and/or carbohydrate) used by the body of the subject during performance of physical or athletic activity, upon administration to the subject of a performance enhancing material and/or upon contact of the subject with a performance enhancing material and/or upon implementation of a performance enhancing activity by the subject.

In further embodiments, the present invention provides a method for measuring an improvement in the ability of a subject to perform a cognitive activity upon administration to the subject of a performance enhancing material and/or upon contact of the subject with a performance enhancing material and/or upon implementation of a performance enhancing activity by the subject, comprising: a) measuring an amount of a biomarker in a first sample of a test subject prior to performance of the cognitive activity; b) measuring the amount of a biomarker in a first sample of a control subject prior to performance of the cognitive activity; c) administering to the subject the performance enhancing material and/or contacting the subject with the performance enhancing material and/or implementing the performance enhancing activity to the test subject; d) having the test subject and control subject perform the cognitive activity; e) measuring the amount of the biomarker in a second sample of the test subject and measuring the amount of the biomarker in a second sample of the control subject after step (d); f) comparing the amount of the biomarker in the first sample and the second sample of the test subject and comparing the amount of the biomarker in the first sample and the second sample of the control subject; and g) comparing the change in the amount of the biomarker of step (e) in the test subject with the change in the amount of the biomarker of step (e) in the control subject, whereby an increase in the amount of biomarker in the test subject that is greater than an increase in the amount of the biomarker in the control subject detects an improvement in the ability of the test subject to perform a cognitive activity upon administration to the subject of a performance enhancing material and/or upon contact of the subject with a performance enhancing material and/or upon implementation of a performance enhancing activity by the subject.

Further provided is a method of detecting a decrement in the performance of a physical or athletic activity in a subject upon administration to the subject of a performance enhancing material and/or upon contact of the subject with a performance enhancing material and/or upon implementation of a performance enhancing activity by the subject, comprising: a) measuring an amount of a biomarker in a first sample of a test subject prior to performance of the physical or athletic activity; b) measuring the amount of a biomarker in a first sample of a control subject prior to performance of the physical or athletic activity; c) administering to the subject the performance enhancing material and/or contacting the subject with the performance enhancing material and/or implementing the performance enhancing activity to the test subject; d) having the test subject and control subject perform the physical or athletic activity; e) measuring the amount of the biomarker in a second sample of the test subject and measuring the amount of the biomarker in a second sample of the control subject after step (d); f) comparing the amount of the biomarker in the first sample and the second sample of the test subject and comparing the amount of the biomarker in the first sample and the second sample of the control subject; and

g) comparing the change in the amount of the biomarker of step (e) in the test subject with the change in the amount of the biomarker of step (e) in the control subject, whereby an increase in the amount of biomarker in the test subject that is less than an increase in the amount of the biomarker in the control subject detects a decrement in the performance of the physical or athletic activity in the subject upon administration to the subject of the performance enhancing material and/or contacting the subject with the performance enhancing material and/or upon implementation of a performance enhancing activity by the subject.

Further provided herein is a method for measuring a decrement in the ability of a subject to perform a cognitive activity upon administration to the subject of a performance enhancing material and/or upon contact of the subject with a performance enhancing material and/or upon implementation of a performance enhancing activity by the subject, comprising: a) measuring an amount of a biomarker in a first sample of a test subject prior to performance of the cognitive activity; b) measuring the amount of a biomarker in a first sample of a control subject prior to performance of the cognitive activity; c) administering to the subject the performance enhancing material and/or contacting the subject with the performance enhancing material and/or implementing the performance enhancing activity to the test subject; d) having the test subject and control subject perform the cognitive activity; e) measuring the amount of the biomarker in a second sample of the test subject and measuring the amount of the biomarker in a second sample of the control subject after step (d); f) comparing the amount of the biomarker in the first sample and the second sample of the test subject and comparing the amount of the biomarker in the first sample and the second sample of the control subject; and g) comparing the change in the amount of the biomarker of step (e) in the test subject with the change in the amount of the biomarker of step (e) in the control subject, whereby an increase in the amount of biomarker in the test subject that is less than an increase in the amount of the biomarker in the control subject detects a decrement in the ability of the test subject to perform a cognitive activity upon administration to the subject of a performance enhancing material and/or upon contact of the subject with a performance enhancing material and/or upon implementation of a performance enhancing activity by the subject.

In additional embodiments, the present invention provides a method for identifying an improvement in the performance of a physical or athletic activity by a subject over time upon administration to the subject of a performance enhancing material and/or upon contact of the subject with a performance enhancing material and/or upon implementation of a performance enhancing activity by the subject, comprising: a) measuring an amount of a biomarker in a first sample of a test subject prior to performance of the physical or athletic activity; b) administering to the subject the performance enhancing material and/or contacting the subject with the performance enhancing material and/or implementing the performance enhancing activity to the test subject at a first time point; c) having the test subject perform the physical or athletic activity at a first time point; d) measuring the amount of the biomarker in a second sample of the test subject after step (c); e) administering to the subject the performance enhancing material and/or contacting the subject with the performance enhancing material and/or implementing the performance enhancing activity to the test subject at a second time point; f) having the test subject perform the physical or athletic activity at a second time point; g) measuring the amount of the biomarker in a third sample of the test subject after step (f); h) comparing the amount of the biomarker in the first sample and the second sample of the test subject; i) comparing the amount of the biomarker in the first sample and the third sample of the test subject; and j) comparing the change in the amount of the biomarker of step (h) in the test subject with the change in the amount of the biomarker of step (i) in the test subject, whereby an increase in the amount of biomarker of step (i) that is greater than an increase in the amount of the biomarker of step (h) identifies an improvement in the performance of a physical or athletic activity in the subject over time upon administration to the subject of a performance enhancing material and/or upon contact of the subject with a performance enhancing material and/or upon implementation of a performance enhancing activity by the subject.

Further provided herein is a method for identifying an improvement in the performance of a cognitive activity in a subject over time upon administration to the subject of a performance enhancing material and/or upon contact of the subject with a performance enhancing material and/or upon implementation of a performance enhancing activity by the subject, comprising: a) measuring an amount of a biomarker in a first sample of a test subject prior to performance of the cognitive activity; b) administering to the subject the performance enhancing material and/or contacting the subject with the performance enhancing material and/or implementing the performance enhancing activity to the test subject at a first time point; c) having the test subject perform the cognitive activity at a first time point; d) measuring the amount of the biomarker in a second sample of the test subject after step (c); e) administering to the subject the performance enhancing material and/or contacting the subject with the performance enhancing material and/or implementing the performance enhancing activity to the test subject at a second time point; f) having the test subject perform the cognitive activity at a second time point; g) measuring the amount of the biomarker in a third sample of the test subject after step (f); h) comparing the amount of the biomarker in the first sample and the second sample of the test subject; i) comparing the amount of the biomarker in the first sample and the third sample of the test subject; and j) comparing the change in the amount of the biomarker of step (h) in the test subject with the change in the amount of the biomarker of step (i) in the test subject, whereby an increase in the amount of biomarker of step (i) that is greater than an increase in the amount of the biomarker of step (h) identifies an improvement in the performance of a cognitive activity in the subject over time upon administration to the subject of a performance enhancing material and/or upon contact of the subject with a performance enhancing material and/or upon implementation of a performance enhancing activity by the subject.

Also provided herein is a method for identifying an improvement in the performance of a physical or athletic activity by a subject upon administration to the subject of a performance enhancing material and/or upon contact of the subject with a performance enhancing material and/or upon implementation of a performance enhancing activity by the subject, comprising: a) measuring an amount of a biomarker in a first sample of a test subject prior to performance of the physical or athletic activity; b) having the test subject perform the physical or athletic activity; c) measuring the amount of the biomarker in a second sample of the test subject of step (b); d) measuring an amount of the biomarker in a third sample of the test subject prior to performance of the physical activity; e) administering to the subject the performance enhancing material and/or contacting the subject with the performance enhancing material and/or having the test subject implement the performance enhancing activity; f) having the test subject perform the same physical or athletic activity as in step (b); g) measuring the amount of the biomarker in a fourth sample of the test subject after step (f); h) comparing the amount of the biomarker in the first sample and the second sample of the test subject; i) comparing the amount of the biomarker in the third sample and the fourth sample of the test subject; and j) comparing the change in the amount of the biomarker of step (h) in the test subject with the change in the amount of the biomarker of step (i) in the test subject, whereby an increase in the amount of biomarker of step (i) that is greater than an increase in the amount of the biomarker of step (h) identifies an improvement in the performance of a physical or athletic activity in the subject upon administration to the subject of a performance enhancing material and/or upon contact of the subject with a performance enhancing material and/or upon implementation of a performance enhancing activity by the subject.

In additional embodiments, the present invention also provides a method for identifying an improvement in the performance of a cognitive activity by a subject upon administration to the subject of a performance enhancing material and/or upon contact of the subject with a performance enhancing material and/or upon implementation of a performance enhancing activity by the subject, comprising: a) measuring an amount of a biomarker in a first sample of a test subject prior to performance of the cognitive activity; b) having the test subject perform the cognitive activity; c) measuring the amount of the biomarker in a second sample of the test subject of step (b); d) measuring an amount of the biomarker in a third sample of the test subject prior to performance of the cognitive activity; e) administering to the subject the performance enhancing material and/or contacting the subject with the performance enhancing material and/or having the test subject implement the performance enhancing activity; f) having the test subject perform the same cognitive activity as in step (b); g) measuring the amount of the biomarker in a fourth sample of the test subject after step (f); h) comparing the amount of the biomarker in the first sample and the second sample of the test subject; i) comparing the amount of the biomarker in the third sample and the fourth sample of the test subject; and j) comparing the change in the amount of the biomarker of step (h) in the test subject with the change in the amount of the biomarker of step (i) in the test subject, whereby an increase in the amount of biomarker of step (i) that is greater than an increase in the amount of the biomarker of step (h) identifies an improvement in the performance of a physical or athletic activity in the subject upon administration to the subject of a performance enhancing material and/or upon contact of the subject with a performance enhancing material and/or upon implementation of a performance enhancing activity by the subject.

It would be well understood that an increase in a biomarker of this invention as detected according to the methods described herein can mean either an improvement in the ability of a subject to perform a physical or athletic activity and/or in the ability to perform a cognitive activity or a decrement in the ability of a subject to perform a physical or athletic activity and/or in the ability to perform a cognitive activity. It is also understood that a decrease in a biomarker of this invention as detected according to the methods described herein can mean either an improvement in the ability of a subject to perform a physical or athletic activity and/or in the ability to perform a cognitive activity or a decrement in the ability of a subject to perform a physical or athletic activity and/or in the ability to perform a cognitive activity.

In the methods of the present invention, the performance enhancing material can be, but is not limited to, a dietary or nutritional supplement or food that comprises or consists primarily of sugars and carbohydrates; a nutritional supplement that contains fenugreek or fenugreek extracts; a nutritional supplement that is composed in part of antioxidants including vitamins C and E; green tea extract; an extract of the Aloe vera plant; melatonin; essential, non-essential and chemically modified amino acids; selenium; cobalt; magnesium; 1-alpha-carnitine; amphetamine; modafinil; creatinine; a dietary supplement composed principally of proteins including whey protein and yeast protein; anabolic steroids; and/or erythropoietin, ephedrine; ginseng; caffeine; forskohlin; glutamine, arginine, whey protein; soy protein; egg albumin; casein; (nutritional compound described in U.S. Pat. No. 6,051,236 as a dry powder comprising protein, amino acids, CHO, vitamins C and E); growth hormone; vitamins, minerals; essential fatty acids; tricarboxylic acid cycle intermediates; free fatty acids; pyruvate; creatine; ipriflavone; pentose (e.g., a monosaccharide such as ribose, D-ribose, ribulose, xylitol, xylulose, any five carbon precursor of ribose; cortisol blockers (e.g., phosphatidylserine, HMB, DHEA, CLA, anabolic steroids, creatine monohydrates, pregnenalone, Ipriflavone, super physiological levels of leucine, anabolic steroids, antioxidants, leucine metabolites, glutamic acid and its metabolites, glutamine and androstenedione).

Furthermore, in the methods of this invention, the performance enhancing activity can be but is not limited to an exercise regimen that incorporates strength training and/or aerobic exercise; massage therapy; meditation; biofeedback; hypnosis; blood doping; isometrics, yoga, acupuncture; aromatherapy; device that emit electromagnetic energy including light and magnetism; and/or counseling.

In addition, in the methods of this invention, the performance enhancing material can be but is not limited to a device that emit photons in the near infra-red spectrum to increase energy output in muscles; clothing, including pants, shorts, socks, briefs, underwear, footwear, orthotics, ankle, knee and leg supports, orthotics and/or other devices designed to increase physical performance and/or reduce fatigue; clothing, wraps, supports and/or other devices that cool muscles; strength training machines/apparatus; endurance machines/apparatus; and/or exercise equipment.

Representative but nonlimiting examples of a biomarker of this invention include a 1365 Dalton protein described in the Examples section herein, interleukin-6, interleukin-1 beta, G-CSF, interferon-gamma, interleukin-8, interleukin-9, MCP-1, MIP-1 beta, TNF alpha, and/or any of the biomarkers listed in Tables 4, 5 and/or 6 in any combination.

Thus, in particular embodiments, the present invention provides a biomarker of this invention, a nucleic acid comprising a nucleotide sequence encoding a biomarker of this invention, a vector comprising said nucleic acid and a cell comprising said vector. The biomarker, the nucleic acid, the vector and/or the cell can be present in a composition comprising a pharmaceutically acceptable carrier.

In some embodiments of this invention, the method can include the measurement of an internal standard from the subject, which can be, for example, an endogenously produced protein, glycoprotein and/or polymer, to normalize the concentration[s] of biomarker[s] measured. This can be done, for example, to correct for relative dilution or other alteration of the body fluids and/or tissues of the subject that are used as the sample in the methods described herein. For example, if there is a 10% change in the internal standard upon carrying out the methods of this invention, then it may be necessary to adjust the values obtained in measurement of the biomarker by 10% to normalize these values. Such changes to the internal standard and the test sample can be due to various physiological conditions that can be present or absent in the subject, including, but not limited to, the level of hydration of the subject, the presence or absence of drugs or other substances in the subject that can exert a physiological effect on the subject, the overall level of fitness of the subject, drugs that reduce the volume of saliva produced, including e.g., anti-cholinergics, and others, drugs that increase the volume of saliva, surgery, radiation, exposure to chemotherapy that may reduce the volume of saliva, and/or other substances, congenital abnormalities that lead to increased or reduced secretion of proteins and/or other substances and/or that lead to a change in biomarker content or water in saliva, and/or bacteria that may produce large amounts of substances, including proteins, that are not associated with those secreted by the body. A nonlimiting list of internal standards of this invention includes alpha-amylase, total protein per unit volume of saliva, sodium, calcium, chloride ion, and/or IgA.

In further embodiments of this invention, a method is provided for identifying a material and/or activity that enhances the performance of a physical or athletic activity and/or the cognitive performance by a subject, comprising: a) measuring an amount of a biomarker in a first sample from a test subject prior to performance of the physical or athletic activity and/or cognitive activity; b) having the test subject perform the physical or athletic activity and/or cognitive activity in the absence of the potentially performance enhancing and/or cognitive performance enhancing material; c) measuring an amount of the biomarker in a second sample from the test subject after step (b); d) administering to the subject the potentially performance enhancing and/or cognitive performance enhancing material and/or contacting the subject with the potentially performance enhancing and/or cognitive enhancing material and/or having the subject implement the potentially performance enhancing and/or cognitive enhancing activity; e) having the test subject perform the physical or athletic activity and or cognitive activity after completion of step (d); f) measuring the amount of the biomarker in a third sample from the test subject after step (e); g) comparing the amount of the biomarker in the first sample and the second sample of the test subject; h) comparing the amount of the biomarker in the first sample and the third sample of the test subject; and i) comparing the change in the amount of the biomarker of step (g) with the change in the amount of the biomarker of step (h) whereby an increase in the amount of biomarker of step (h) that is greater than an increase in the amount of the biomarker of step (g) identifies a material that enhances the performance of a physical or athletic activity and/or cognitive activity by a subject.

In the methods of this invention, the physical and/or athletic activity can be, but is not limited to, running, walking, bicycling, weight lifting, swimming, a standardized physical test course including, for example, those used by the military, triathlons, biathlons, shooting of rifles, shooting of handguns, the aiming of computerized target equipment, staying awake, hiking while carrying a large burden on the back.

Furthermore, in the methods of this invention, the cognitive activity can be, but is not limited to, performance of a Folstein's Mini-Mental State Examination (MMSE), Peabody Picture Vocabulary Test, Peabody Developmental Motor Scale, Stanford-Binet Intelligence Scale, Battelle Developmental Inventory, Vineland Adaptive Behavior Scale, Woodcock-Johnson Psycho-Educational Battery-Revised, Standard and Supplemental Batteries (WJPEB-R), Wechsler Adult Intelligence Scale—Revised or III (WAIS-R or WAIS III), Wechsler Intelligence Scale for Children (WISC-R or WISC-III), Woodcock-Johnson Psycho-Educational Battery, Wechsler Individual Achievement Test (WIAT), Stanford Test of Academic Skills (STAS), Scholastic Abilities Test for Adults (SATA), Detroit tests of Learning Aptitude-Adult 2, 3, Stanford Diagnostic Mathematics Test, Formal Reading Inventory, Woodcock Reading Mastery Tests-Revised, Gray Oral Reading Test, LSAT, SAT, GMAT, GRE and/or computerized test batteries that measure response time, ability to perform standardized calculations, tasks to measure response time to a visual cue, scores on various computer games and other tasks, on a computerized instrument including handheld devices, PDAs, laptops and desktop computers.

Also provided herein as an additional embodiment is a method of correlating an increase in the amount of a biomarker in a subject with an improvement in the performance of a physical or athletic activity by the subject upon administration to the subject of a performance enhancing material and/or upon contact of the subject with a performance enhancing material and/or upon implementation of a performance enhancing activity by the subject, comprising: a) detecting in the subject an improvement in the performance of a physical or athletic activity upon administration to the subject of a performance enhancing material and/or upon contact of the subject with a performance enhancing material and/or upon implementation of a performance enhancing activity by the subject by measuring exhaled gases; a change in body composition, including percentage body fat, or percentage muscle; a maximum work output as measured on standard tests including cycle ergometers, treadmills or other machines where energy expenditure over a period of time or maximum output can be measured; and/or a reduction in the amount of time required to perform a standardized physical task including the time required to run, hike, bike, swim or walk a known distance, etc; b) detecting in the subject of (a) an increase in the amount of a biomarker in the subject; and c) correlating the increase in the amount of the biomarker of step (b) with an improvement in the performance of a physical or athletic activity in the subject upon administration to the subject of a performance enhancing material and/or upon contact of the subject with a performance enhancing material and/or upon implementation of a performance enhancing activity by the subject.

In addition, the present invention provides a method of correlating an increase in the amount of a biomarker in a subject with an improvement in the performance of a cognitive activity by the subject upon administration to the subject of a performance enhancing material and/or upon contact of the subject with a performance enhancing material and/or upon implementation of a performance enhancing activity by the subject, comprising:

a) detecting in the subject an improvement in the performance of a cognitive activity upon administration to the subject of a performance enhancing material and/or upon contact of the subject with a performance enhancing material and/or upon implementation of a performance enhancing activity by the subject by measuring cognitive activity upon administration of, for example, Folstein's Mini-Mental State Examination (MMSE), Peabody Picture Vocabulary Test, Peabody Developmental Motor Scale, Stanford-Binet Intelligence Scale, Battelle Developmental Inventory, Vineland Adaptive Behavior Scale, Woodcock-Johnson Psycho-Educational Battery-Revised, Standard and Supplemental Batteries (WJPEB-R), Wechsler Adult Intelligence Scale—Revised or III (WAIS-R or WAIS III), Wechsler Intelligence Scale for Children (WISC-R or WISC-III), Woodcock-Johnson Psycho-Educational Battery, Wechsler Individual Achievement Test (WIAT), Stanford Test of Academic Skills (STAS), Scholastic Abilities Test for Adults (SATA), Detroit tests of Learning Aptitude-Adult 2, 3, Stanford Diagnostic Mathematics Test, Formal Reading Inventory, Woodcock Reading Mastery Tests-Revised, Gray Oral Reading Test, LSAT, SAT, GMAT, GRE and/or computerized test batteries that measure response time, ability to perform standardized calculations, tasks to measure response time to a visual cue, scores on various computer games and other tasks, on a computerized instrument including handheld devices, PDAs, laptops and desktop computers; b) detecting in the subject of (a) an increase in the amount of a biomarker in the subject; and c) correlating the increase in the amount of the biomarker of step (b) with an improvement in the performance of a cognitive activity in the subject upon administration to the subject of a performance enhancing material and/or upon contact of the subject with a performance enhancing material and/or upon implementation of a performance enhancing activity by the subject.

Also provided herein is a method of correlating a decrease in the amount of a biomarker in a subject with a decrement in the performance of a physical or athletic activity by the subject upon administration to the subject of a performance enhancing material and/or upon contact of the subject with a performance enhancing material and/or upon implementation of a performance enhancing activity by the subject, comprising: a) detecting in the subject a decrement in the performance of a physical or athletic activity upon administration to the subject of a performance enhancing material and/or upon contact of the subject with a performance enhancing material and/or upon implementation of a performance enhancing activity by the subject by measuring exhaled gases; a change in body composition, including percentage body fat, or percentage muscle; a maximum work output as measured on standard tests including cycle ergometers, treadmills or other machines where energy expenditure over a period of time or maximum output can be measured; and/or a change in the amount of time required to perform a standardized physical task including the time required to run, hike, bike, swim or walk a known distance, etc; b) detecting in the subject of (a) a decrease in the amount of a biomarker in the subject; and c) correlating the decrease in the amount of the biomarker of step (b) with a decrement in the performance of a physical or athletic activity in the subject upon administration to the subject of a performance enhancing material and/or upon contact of the subject with a performance enhancing material and/or upon implementation of a performance enhancing activity by the subject.

In addition, the present invention provides a method of correlating a decrease in the amount of a biomarker in a subject with a decrement in the performance of a cognitive activity by the subject upon administration to the subject of a performance enhancing material and/or upon contact of the subject with a performance enhancing material and/or upon implementation of a performance enhancing activity by the subject, comprising: a) detecting in the subject a decrement in the performance of a cognitive activity upon administration to the subject of a performance enhancing material and/or upon contact of the subject with a performance enhancing material and/or upon implementation of a performance enhancing activity by the subject by measuring cognitive activity upon administration of, for example, Folstein's Mini-Mental State Examination (MMSE), Peabody Picture Vocabulary Test, Peabody Developmental Motor Scale, Stanford-Binet Intelligence Scale, Battelle Developmental Inventory, Vineland Adaptive Behavior Scale, Woodcock-Johnson Psycho-Educational Battery-Revised, Standard and Supplemental Batteries (WJPEB-R), Wechsler Adult Intelligence Scale—Revised or III (WAIS-R or WAIS III), Wechsler Intelligence Scale for Children (WISC-R or WISC-III), Woodcock-Johnson Psycho-Educational Battery, Wechsler Individual Achievement Test (WIAT), Stanford Test of Academic Skills (STAS), Scholastic Abilities Test for Adults (SATA), Detroit tests of Learning Aptitude-Adult 2, 3, Stanford Diagnostic Mathematics Test, Formal Reading Inventory, Woodcock Reading Mastery Tests-Revised, Gray Oral Reading Test, LSAT, SAT, GMAT, GRE and/or computerized test batteries that measure response time, ability to perform standardized calculations, tasks to measure response time to a visual cue, scores on various computer games and other tasks, on a computerized instrument including handheld devices, PDAs, laptops and desktop computers; b) detecting in the subject of (a) a decrease in the amount of a biomarker in the subject; and c) correlating the decrease in the amount of the biomarker of step (b) with a decrement in the performance of a cognitive activity in the subject upon administration to the subject of a performance enhancing material and/or upon contact of the subject with a performance enhancing material and/or upon implementation of a performance enhancing activity by the subject.

Further provided herein is a method of identifying an improvement in the performance of a physical or athletic activity in a subject upon administration to the subject of a performance enhancing material and/or upon contact of the subject with a performance enhancing material and/or upon implementation of a performance enhancing activity by the subject, comprising: a) correlating an increase in the amount of a biomarker in the subject with an improvement in the performance of a physical or athletic activity in the subject upon administration to the subject of a performance enhancing material and/or upon contact of the subject with a performance enhancing material and/or upon implementation of a performance enhancing activity by the subject; and b) detecting an increase in the biomarker of step (a) in the subject, thereby identifying an improvement in the performance of a physical or athletic activity in the subject upon administration to the subject of a performance enhancing material and/or upon contact of the subject with a performance enhancing material and/or upon implementation of a performance enhancing activity by the subject

The present invention further provides a method of identifying an improvement in the performance of a cognitive activity in a subject upon administration to the subject of a performance enhancing material and/or upon contact of the subject with a performance enhancing material and/or upon implementation of a performance enhancing activity by the subject, comprising: a) correlating an increase in the amount of a biomarker in the subject with an improvement in the performance of a cognitive activity in the subject upon administration to the subject of a performance enhancing material and/or upon contact of the subject with a performance enhancing material and/or upon implementation of a performance enhancing activity by the subject; and b) detecting an increase in the biomarker of step (a) in the subject, thereby identifying an improvement in the performance of a cognitive activity in the subject upon administration to the subject of a performance enhancing material and/or upon contact of the subject with a performance enhancing material and/or upon implementation of a performance enhancing activity by the subject.

The present invention also provides a method of identifying a decrement in the performance of a physical or athletic activity in a subject upon administration to the subject of a performance enhancing material and/or upon contact of the subject with a performance enhancing material and/or upon implementation of a performance enhancing activity by the subject, comprising: a) correlating a decrease in the amount of a biomarker in the subject with a decrement in the performance of a physical or athletic activity in the subject upon administration to the subject of a performance enhancing material and/or upon contact of the subject with a performance enhancing material and/or upon implementation of a performance enhancing activity by the subject; and b) detecting a decrease in the biomarker of step (a) in the subject, thereby identifying a decrement in the performance of a physical or athletic activity in the subject upon administration to the subject of a performance enhancing material and/or upon contact of the subject with a performance enhancing material and/or upon implementation of a performance enhancing activity by the subject.

Furthermore, the present invention provides a method of identifying a decrement in the performance of a cognitive activity in a subject upon administration to the subject of a performance enhancing material and/or upon contact of the subject with a performance enhancing material and/or upon implementation of a performance enhancing activity by the subject, comprising: a) correlating a decrease in the amount of a biomarker in the subject with a decrement in the performance of a cognitive activity in the subject upon administration to the subject of a performance enhancing material and/or upon contact of the subject with a performance enhancing material and/or upon implementation of a performance enhancing activity by the subject; and b) detecting a decrease in the biomarker of step (a) in the subject, thereby identifying a decrement in the performance of a physical or athletic activity in the subject upon administration to the subject of a performance enhancing material and/or upon contact of the subject with a performance enhancing material and/or upon implementation of a performance enhancing activity by the subject.

The biomarkers of this invention are correlated with an improvement or decrement in the performance of a physical and/or athletic activity and/or in the performance of a cognitive activity as described herein according to methods well known in the art and as disclosed in the Examples provided herein. In general, identifying such correlation involves conducting analyses that establish a statistically significant association and/or a statistically significant correlation between the presence of a biomarker or a combination of biomarkers and a change (e.g., an improvement or decrement) in the performance of a physical and/or athletic activity and/or in the performance of a cognitive activity as detected according to standard methods. An analysis that identifies a statistical association (e.g., a significant association) between the biomarker or combination of biomarkers and the improvement or decrement establishes a correlation between the increase or decrease of the biomarker or combination of biomarkers in a subject and the activity being analyzed.

It would be well understood by one of skill in the art that the methods of the present invention can be carried out on multiple subjects and the data compiled to produce mean and median values that indicate improvement or decrement in the ability to perform an physical or athletic activity and/or a cognitive activity of this invention. It would also be under stood that the statistical limits described by the data obtained from groups of subjects can be applied to individual subjects' response. Thus, it would be understood that in some embodiments of this invention, the methods of identifying an improvement or decrement in the ability to perform an physical or athletic activity and/or to perform a cognitive activity can be carried out using a computer database, wherein the data from multiple subjects are stored in a computer database and analyzed according to art known methods of statistical and mathematical analysis to identify means, medians, trends, statistically significant changes, variances, etc.

In some embodiments, the methods of correlating biomarkers with changes in physical/athletic performance and/or cognitive performance can be carried out using a computer database. Thus the present invention provides a computer-assisted method of identifying a proposed performance-enhancing material and/or activity. The method involves the steps of (a) storing a database of biological data for a plurality of subjects, the biological data that is being stored including for each of said plurality of subjects: (i) a description of the physical/athletic and/or cognitive activity performed by the subject, (ii) a description of the performance enhancing material and/or activity administered to, contacted with and/or implemented by the subject; (iii) a description of measurements according to art-known methods detecting a change in performance in the subject; and (iv) a description of measurements of biomarkers in the subject; and then (b) querying the database to determine the relationship between a change in the measurement of biomarkers in the subject and change in performance of the subject. Such querying can be carried out prospectively or retrospectively on the database by any suitable means, but is generally done by statistical analysis in accordance with known techniques, as described herein.

In the methods of this invention employing measurements over time, the time intervals can be minutes, hours, days, weeks, months and/or years.

In the methods of this invention involving the detection of a decrement in the performance of a physical or athletic activity and/or a decrement in the performance of a cognitive activity, such methods can be used to identify a subject having, for example, a disease state or other pathological state, an injury, a genetic disorder or defect, a toxic or intoxicated state, etc.

In the methods of this invention, the sample can be any biological fluid or tissue that can be used in an assay of this invention, including but not limited to, serum, plasma, blood, saliva, semen, lymph, cerebrospinal fluid, prostatic fluid, urine, sputum, joint fluid, body cavity fluid, tear fluid, anal secretions; vaginal secretions, perspiration, whole cells, cell extracts, tissue, biopsy material, aspirates, exudates, slide preparations, fixed cells, tissue sections, etc.

A subject of this invention is any mammal capable of performing physical and/or athletic activity and for which detection of a change in physical and/or athletic performance is desired. In some embodiments, the subject is human. In other embodiments, the subject can be a horse, a dog.

The term “antibody” as used herein, includes, but is not limited to a polypeptide encoded by an immunoglobulin gene or immunoglobulin genes, or a fragment thereof. “Antibody” also includes, but is not limited to, a polypeptide encoded by an immunoglobulin gene or immunoglobulin genes, or a fragment thereof, which specifically binds to and recognizes the biomarkers of this invention.

The term “epitope” means an antigenic determinant that is specifically bound by an antibody. Epitopes usually consist of surface groupings of molecules such as amino acids and/or sugar side chains and usually have specific three-dimensional structural characteristics, as well as specific charge characteristics.

The terms “specifically binds to” and “specifically reactive with” refer to a binding reaction that is determinative of the presence of the antigen and antibody or aptamer and target in the presence of a heterogeneous population of proteins, nucleic acids and/or other biologics. Thus, under designated assay conditions, the specified antibodies and antigens and/or aptamers and targets bind to one another and do not bind in a significant amount to other components present in a sample.

In some embodiments employing antibodies, a variety of immunoassay formats can be used to select antibodies specifically reactive with a particular antigen. For example, solid-phase ELISA immunoassays are routinely used to select monoclonal antibodies specifically immunoreactive with an analyte. See Harlow and Lane (ANTIBODIES: A LABORATORY MANUAL, Cold Springs Harbor Publications, New York, (1988)) for a description of immunoassay formats and conditions that can be used to determine specific immunoreactivity. Typically a specific or selective reaction will be at least twice background signal to noise and more typically more than 10 to 100 times greater than background.

An “immunologically reactive fragment” of a protein refers to a portion of the protein or peptide that is immunologically reactive with a binding partner, e.g., an antibody, which is immunologically reactive with the protein itself.

Antibodies to biomarkers of this invention can be generated using methods that are well known in the art. Such antibodies can include, but are not limited to, polyclonal, monoclonal, chimeric, humanized, single chain, Fab fragments, and/or fragments produced by an expression library, including e.g., phage display. (See, e.g., Paul, FUNDAMENTAL IMMUNOLOGY, 3rd Ed., 1993, Raven Press, New York, for antibody structure and terminology.)

Antibody fragments that contain specific binding sites for a biomarker of this invention can also be generated. For example, such fragments include, but are not limited to, the F(ab′)₂ fragments that can be produced by pepsin digestion of the antibody molecule, and the Fab fragments that can be generated by reducing the disulfide bridges of the F(ab′)₂ fragments. Alternatively, Fab expression libraries can be constructed to allow rapid and easy identification of monoclonal Fab fragments with the desired specificity (Huse et al., Science 254, 1275-1281 (1989)).

For the production of antibodies, various hosts including goats, rabbits, rats, mice, humans, and others, may be immunized by injection with a protein or any fragment or oligopeptide or conjugate thereof that has immunogenic properties. Depending on the host species, various adjuvants can be used to increase the immunological response. Such adjuvants include, but are not limited to, Freund's complete and incomplete adjuvant, mineral gels such as aluminum hydroxide, and surface active substances such as lysolecithin, pluronic polyols, polyanions, peptides, oil emulsions, keyhole limpet hemocyanin, and dinitrophenol. Examples of adjuvants used in humans include BCG (bacilli Calmette-Guerin) and Corynebacterium parvum.

Monoclonal antibodies can be prepared using any technique that provides for the production of antibody molecules by continuous cell lines in culture. These include, but are not limited to, the hybridoma technique, the human B-cell hybridoma technique, and the EBV-hybridoma technique (Kohler et al. (1975) Nature 256:495-497; Kozbor et al. (1985) J. Immunol. Methods 81:31-42; Cote et al. (1983) Proc. Natl. Acad. Sci. 80:2026-2030; Cole et al. (1984) Mol. Cell. Biol. 62:109-120). Briefly, the procedure can be as follows: an animal is immunized with a protein or immunogenic fragment or oligopeptide or conjugate thereof. Lymphoid cells (e.g., splenic lymphocytes) are then obtained from the immunized animal and fused with immortalizing cells (e.g., myeloma or heteromyeloma) to produce hybrid cells. The hybrid cells are screened to identify those that produce the desired antibody.

Human hybridomas that secrete human antibody can be produced by the Kohler and Milstein technique and according to art-known protocols. Hybridoma production in rodents, especially mouse, is a very well established procedure and thus, stable murine hybridomas provide an unlimited source of antibody of select characteristics. As an alternative to human antibodies, the mouse antibodies can be converted to chimeric murine/human antibodies by genetic engineering techniques. See Oi et al., Bio Techniques 4(4):214-221 (1986); Sun et al., Hybridoma 5 (1986).

The monoclonal antibodies of this invention specific for biomarker protein epitopes of this invention can also be used to produce anti-idiotypic (paratope-specific) antibodies. (See e.g., McNamara et al., Science 220, 1325-26 (1984); Kennedy et al., Science 232:220 (1986).) These antibodies resemble the biomarker protein epitope and thus can be used as an antigen to stimulate an immune response against the biomarker protein.

In addition, techniques developed for the production of “chimeric antibodies,” the splicing of mouse antibody genes to human antibody genes to obtain a molecule with appropriate antigen specificity and biological activity can be used (Morrison et al. Proc. Natl. Acad. Sci. 81:6851-6855 (1984); Neuberger et al., Nature 312:604-608 (1984); Takeda et al., Nature 314:452-454 (1985)). Alternatively, techniques described for the production of single chain antibodies can be adapted, using methods known in the art, to produce biomarker protein-specific single chain antibodies. Antibodies with related specificity, but of distinct idiotypic composition, can be generated by chain shuffling from random combinatorial immunoglobin libraries (Burton, Proc. Natl. Acad. Sci. 88:11120-3 (1991)).

Antibodies can also be produced by inducing in vivo production in the lymphocyte population or by screening immunoglobulin libraries or panels of highly specific binding reagents as described in the literature (Orlandi et al., Proc. Natl. Acad. Sci. 86:3833-3837 (1989)); Winter et al., Nature 349:293-299 (1991)).

Various immunoassays can be used to identify biomarkers of this invention. Such immunoassays typically involve the measurement of antigen/antibody complex formation between a biomarker protein or peptide and its specific antibody.

The immunoassays of the invention can be either competitive or noncompetitive and both types of assays are well-known and well-developed in the art. In competitive binding assays, antigen or antibody competes with a detectably labeled antigen or antibody for specific binding to a capture site bound to a solid surface. The concentration of labeled antigen or antibody bound to the capture agent is inversely proportional to the amount of free antigen or antibody present in the sample.

Noncompetitive assays of this invention can be sandwich assays, in which, for example, the antigen is bound between two antibodies. One of the antibodies is used as a capture agent and is bound to a solid surface. The other antibody is labeled and is used to measure or detect the resultant antigen/antibody complex by e.g., visual or instrument means. A number of combinations of antibody and labeled antibody can be used, as are well known in the art. In some embodiments, the antigen/antibody complex can be detected by other proteins capable of specifically binding human immunoglobulin constant regions, such as protein A, protein L or protein G. These proteins are normal constituents of the cell walls of streptococcal bacteria. They exhibit a strong nonimmunogenic reactivity with immunoglobulin constant regions from a variety of species. (See, e.g., Kronval et al., J. Immunol., 111:1401-1406 (1973); Akerstrom et al., J. Immunol., 135:2589-2542 (1985).)

In some embodiments, the non-competitive assays need not be sandwich assays. For instance, the antibodies or antigens in the sample can be bound directly to the solid surface. The presence of antibodies or antigens in the sample can then be detected using labeled antigen or antibody, respectively.

In some embodiments, antibodies and/or proteins can be conjugated or otherwise linked or connected (e.g., covalently or noncovalently) to a solid support (e.g., bead, plate, slide, dish, membrane or well) in accordance with known techniques. Antibodies can also be conjugated or otherwise linked or connected to detectable groups such as radiolabels (e.g., ³⁵S, ¹²⁵I, ³²p, ¹³H, ¹⁴C, ¹³¹I), enzyme labels (e.g., horseradish peroxidase, alkaline phosphatase), gold beads, chemiluminescence labels, ligands (e.g., biotin) and/or fluorescence labels (e.g., fluorescein) in accordance with known techniques.

A variety of organic and inorganic polymers, both natural and synthetic can be used as the material for the solid surface. Nonlimiting examples of polymers include polyethylene, polypropylene, poly(4-methylbutene), polystyrene, polymethacrylate, poly(ethylene terephthalate), rayon, nylon, poly(vinyl butyrate), polyvinylidene difluoride (PVDF), silicones, polyformaldehyde, cellulose, cellulose acetate, nitrocellulose, and the like. Other materials that can be used include, but are not limited to, include paper, glass, ceramic, metal, metalloids, semiconductive materials, cements and the like. In addition, substances that form gels, such as proteins (e.g., gelatins), lipopolysaccharides, silicates, agarose and polyacrylamides can be used. Polymers that form several aqueous phases, such as dextrans, polyalkylene glycols or surfactants, such as phospholipids, long chain (12-24 carbon atoms) alkyl ammonium salts and the like are also suitable. Where the solid surface is porous, various pore sizes can be employed depending upon the nature of the system.

A variety of immunoassay systems can be used, including but not limited to, radio-immunoassays (RIA), enzyme-linked immunosorbent assays (ELISA) assays, enzyme immunoassays (EIA), “sandwich” assays, gel diffusion precipitation reactions, immunodiffusion assays, agglutination assays, immunofluorescence assays, fluorescence activated cell sorting (FACS) assays, immunohistochemical assays, protein A immunoassays, protein G immunoassays, protein L immunoassays, biotin/avidin assays, biotin/streptavidin assays, immunoelectrophoresis assays, precipitation/flocculation reactions, immunoblots (Western blot; dot/slot blot); immunodiffusion assays; liposome immunoassay, chemiluminescence assays, library screens, expression arrays, etc., immunoprecipitation, competitive binding assays and immunohistochemical staining. These and other assays are described, among other places, in Hampton et al. (Serological Methods, a Laboratory Manual, APS Press, St Paul, Minn. (1990)) and Maddox et al. (J. Exp. Med. 158:1211-1216 (1993); the entire contents of which are incorporated herein by reference for teachings directed to immunoassays).

The methods of this invention can also be carried out using a variety of solid phase systems, such as described in U.S. Pat. No. 5,879,881, as well as in a dry strip lateral flow system (e.g., a “dipstick” system), such as described, for example, in U.S. Patent Publication No. 20030073147, the entire contents of each of which are incorporated by reference herein.

In some embodiments, the biomarker of this invention can be detected and/or quantitated in an assay employing a peptide aptamer, which binds to the biomarker in a manner similar to an antibody, a ligand or a small molecule.

As used herein, the term “aptamer” includes any small peptide or nucleic acid molecule that specifically recognizes and binds a protein (e.g., a target protein such as a biomolecule of this invention). In some embodiments, a peptide aptamer of this invention can be a recombinant protein or peptide that has been selected for specific binding to a target protein according to art-known methods (see, e.g., Hoppe-Seyler, Crnkovic-Mertens et al. 2004). This can be a short peptide domain inserted into a supporting protein scaffold that enhances both specificity and affinity by conformationally constraining the peptide sequence (Colas, Cohen et al. 1996; Cohen, Colas et al. 1998; Buerger, Nagel-Wolfrum et al. 2003). In some embodiments of the present invention employing a peptide aptamer, the term “aptamer” can be used to designate the peptide in the scaffold protein while the term “peptide” can refer to the inserted sequence.

In other embodiments, an aptamer can be a small, usually stabilized, nucleic acid molecule that comprises a binding domain for a target molecule (e.g., a biomarker of this invention). Nucleic acid aptamers of this invention are DNA or RNA molecules that have been selected from random pools based on their ability to bind nucleic acid, proteins, small organic compounds, and even entire organisms, usually with high affinity.

In further embodiments, a nucleic acid aptamer can comprise at least one modified nucleotide base. The term “modified nucleotide base” encompasses nucleotides with a covalently modified base and/or sugar. For example, modified nucleotides include nucleotides having sugars that are covalently attached to low molecular weight organic groups other than a hydroxyl group at the 3′ position and other than a phosphate group at the 5′ position. Such modified nucleotides can also include 2′ substituted sugars such as 2′-O-methyl; 2′-O-alkyl; 2′-O-allyl; 2′-S-alkyl; 2′-S-allyl; 2′-fluoro; 2′-halo; or 2′-azido-ribose, carbocyclic sugar analogues, a-anomeric sugars; epimeric sugars such as arabinose, xyloses or lyxoses, pyranose sugars, furanose sugars, and sedoheptulose.

Modified nucleotides of this invention can include but are not limited to, alkylated purines and/or pyrimidines; acylated purines and/or pyrimidines; and other heterocycles. These classes of pyrimidines and purines are known in the art and include, pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2 methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpseudouracil; 1-methylguanine; and 1-methylcytosine.

Nucleic acid aptamers of this invention can be synthesized from conventional phosphodiester linked nucleotides using standard solid or solution phase synthesis techniques that are known in the art. Linkages between nucleotides can use alternative linking molecules. For example, linking groups of the formula P(O)S, (thioate); P(S)S, (dithioate); P(O)NR12; P(O)R′; P(O)OR6; CO; or CONR12 wherein R is H (or a salt) or alkyl (1-12C) and R6 is alkyl (1-9C) is joined to adjacent nucleotides through —O— or —S—.

In certain embodiments, the present invention can employ monoclonal or polyclonal aptamers. A “monoclonal aptamer” as used herein includes a single aptamer with a known nucleotide sequence. A “polyclonal aptamer” as used herein includes a population of aptamers with the same or different nucleotide sequences that all have an affinity for the same target molecule.

As used herein, the term “signaling aptamer” includes aptamers with reporter molecules, such as a fluorescence dye, attached to a nucleotide in such a way that upon conformational changes resulting from the interaction of the aptamer with a ligand, the reporter molecule yields a differential signal, such as, for example, a change in fluorescence intensity. Examples of signaling aptamers can be found, for example, in U.S. Pat. No. 6,706,481, the entire contents of which are incorporated by reference herein for the disclosure of aptamers, methods of making aptamers and/or methods of using aptamers.

The preparation of aptamers is well known in the art and may involve, inter alia, the use of combinatorial RNA libraries to identify binding sites (Gold, Ann. Rev. Biochem. 64 (1995), 763-797). Aptamers are readily made that bind to a wide variety of molecules. Each of these molecules can be used as a modulator of gene expression using the methods of the invention. For example, organic molecules, nucleotides, amino acids, polypeptides, target features on cell surfaces, metal ions, saccharides, have all been shown to be suitable for isolating aptamers that can specifically bind to the respective ligand. For instance, organic dyes such as Hoechst 33258 have been successfully used as target ligands in vitro aptamer selections (Werstuck and Green, Science 282:296-298 (1998)). Other small organic molecules like dopamine, theophylline, sulforhodamine B, and cellobiose have also been used as ligands in the isolation of aptamers. Aptamers have also been isolated for antibiotics such as kanamycin A, lividomycin, tobramycin, neomycin B, viomycin, chloramphenicol and streptomycin. For a review of aptamers that recognize small molecules, see Famulok, Science 9:324-9 (1999).

In a preferred embodiment, the ligand of the aptamer of the nucleic acid molecule of the invention is a cell-permeable, small organic molecule. Small organic molecules that do not have a general inhibitory effect on translation are preferred as ligands. The small molecule preferably also exhibits in vivo persistence sufficient for achieving the desired level of inhibition of translation. The molecules also can be screened to identify those that are bioavailable after, for example, oral administration. In a preferred embodiment of the invention, the ligand is nontoxic. The ligand may optionally be a drug, including, for example, a steroid. However, in some of the methods of controlling gene expression, it is preferable that the ligand be pharmacologically inert. In some embodiments, the ligand is a polypeptide whose presence in the cell is indicative of a disease or pathological condition.

Thus, the ligand for an aptamer is optionally an antibiotic, such as chloramphenicol. In an alternative embodiment, the ligand of the aptamer is an organic dye such as Hoeschst dye 33258. In still another embodiment, the ligand may be a metal ion.

The aptamer of the nucleic acid of the invention can be comprised entirely of RNA. In other embodiments of the invention, however, the aptamer can instead be comprised entirely of DNA, or partially of DNA, or partially of other nucleotide analogs. To specifically inhibit translation in vivo, RNA aptamers are preferred. Such RNA aptamers are preferably introduced into a cell as a DNA that encodes the aptamer sequence such that transcription results in the RNA aptamer. Alternatively, an RNA aptamer itself can be introduced into a cell.

Aptamers are typically developed to bind particular ligands by employing known in vivo or in vitro (most typically, in vitro) selection techniques known as SELEX (Systematic Evolution of Ligands by Exponential Enrichment). Methods of making aptamers are described in, for example, Ellington and Szostak, Nature 346:818 (1990), Tuerk and Gold, Science 249:505 (1990), U.S. Pat. No. 5,582,981, PCT Publication No. WO 00/20040, U.S. Pat. No. 5,270,163, Lorsch and Szostak, Biochemistry, 33:973 (1994), Mannironi et al., Biochemistry 36:9726 (1997), Blind, Proc. Nat'l. Acad. Sci. USA 96:3606-3610 (1999), Huizenga and Szostak, Biochemistry 34:656-665 (1995), PCT Publication Nos. WO 99/54506, WO 99/27133, WO 97/42317 and U.S. Pat. No. 5,756,291.

Generally, in their most basic form, in vitro selection techniques for identifying RNA aptamers involve first preparing a large pool of DNA molecules of the desired length that contain at least some region that is randomized or mutagenized. For instance, a common oligonucleotide pool for aptamer selection might contain a region of 20-100 randomized nucleotides flanked on both ends by an about 15-25 nucleotide long region of defined sequence useful for the binding of PCR primers. The oligonucleotide pool is amplified using standard PCR techniques. The DNA pool is then transcribed in vitro. The RNA transcripts are then subjected to affinity chromatography. The transcripts are most typically passed through a column or contacted with magnetic beads or the like on which the target ligand has been immobilized. RNA molecules in the pool that bind to the ligand are retained on the column or bead, while nonbinding sequences are washed away. The RNA molecules that bind the ligand are then reverse transcribed and amplified again by PCR (usually after elution). The selected pool sequences are then put through another round of the same type of selection. Typically, the pool sequences are put through a total of about three to ten iterative rounds of the selection procedure. The cDNA is then amplified, cloned, and sequenced using standard procedures to identify the sequence of the RNA molecules that are capable of acting as aptamers for the target ligand.

For use in the present invention, the aptamer is preferably selected for ligand binding in the presence of salt concentrations and temperatures that mimic normal physiological conditions.

Once an aptamer sequence has been successfully identified, the aptamer may be further optimized by performing additional rounds of selection starting from a pool of oligonucleotides comprising the mutagenized aptamer sequence.

One can generally choose a suitable ligand without reference to whether an aptamer is yet available. In most cases, an aptamer can be obtained which binds the small, organic molecule of choice by someone of ordinary skill in the art. The unique nature of the in vitro selection process allows for the isolation of a suitable aptamer that binds a desired ligand despite a complete dearth of prior knowledge as to what type of structure might bind the desired ligand.

For an aptamer to be suitable for use in the present invention, the binding affinity of the aptamer for the ligand must be sufficiently strong and the structure formed by the aptamer when bound to its ligand must be significant enough so as to disrupt translation of the attached transcript. The structure of the aptamer in the absence of the ligand, on the other hand, should be minimal. Whether or not an aptamer meets these criteria can be readily determined by one of ordinary skill in the art. For instance, a chosen aptamer could be inserted in the 5′-UTR of a reporter gene on an expression vector. In the absence of the aptamer's ligand, cells transfected with the expression vector should show expression of the reporter gene. Once the aptamer is added, however, the expression should cease if the aptamer of the mRNA is effective in blocking translation. One of ordinary skill in the art will recognize that effective conditional expression of a reporter gene could be determined in any of a number of cells or animal models and using a number of different available reporter genes such as genes encoding green fluorescent protein (GFP), yellow fluorescent protein (YFP), and blue fluorescent protein (BFP). Other suitable marker genes include the thymidine kinase (tk), dihydrofolate reductase (DHFR), chloramphenicol acetyltransferase (CAT), β-lactamase, β-galactosidase (β-gal), and aminoglycoside phosphotransferase (APH) genes.

The association constant for the aptamer and associated ligand is preferably such that the ligand functions to bind to the aptamer and have the desired effect at the concentration of ligand obtained upon administration of the ligand. For in vivo use, for example, the association constant should be such that binding occurs well below the concentration of ligand that can be achieved in the serum or other tissue. Preferably, the required ligand concentration for in vivo use is also below that which could have undesired effects on the organism.

In the methods of the present invention employing peptide aptamers, assays similar to the immunoassays described herein can be carried out to detect and/or quantify a biomarker of this invention, whereby an aptamer is used in place of an antibody and an aptamer/target molecule complex, rather than an antibody/antigen complex is detected. The immunoassays described herein can also be adapted to employ a nucleic acid aptamer in place of an antibody, for the detection of a nucleic acid/target molecule complex. In some embodiments, the immunoassays of this invention can also be modified to employ both aptamers and antibodies to detect and/or quantify a biomarker of this invention. Modification of any known immunoassay to accommodate the detection of binding of a peptide aptamer or a nucleic acid aptamer to a target molecule would be well known to one of ordinary skill in the art.

In other embodiments of this invention, a nucleic acid having the nucleotide sequence or a substantially similar nucleotide sequence of the gene encoding a biomarker protein of this invention can be used as a probe in a nucleic acid hybridization assay for the detection of a biomarker protein in various tissues and/or body fluids of a subject of this invention. The probe can be used in any type of nucleic acid hybridization assay including Southern blots (Southern, 1975, J. Mol. Biol. 98:508), Northern blots (Thomas et al., 1980, Proc. Nat/Acad. Sci. U.S.A. 77:5201-05), colony blots (Grunstein et al., 1975, Proc. Natl. Acad. Sci. U.S.A. 72:3961-65), slot blots, dot blots, etc. Stringency of hybridization can be varied depending on the requirements of the assay according to methods well known in the art. Assays for detecting nucleic acid encoding a protein in a cell, or the amount thereof, typically involve first contacting the cells or extracts of the cells containing nucleic acids therefrom with an oligonucleotide probe that specifically binds to nucleic acid encoding a protein or peptide as described herein (typically under conditions that permit access of the oligonucleotide to intracellular material), and then detecting the presence or absence of binding of the oligonucleotide probe thereto. Any suitable assay format can be employed (see, e.g., U.S. Pat. No. 4,358,535; U.S. Pat. Nos. 4,302,204; 4,994,373; 4,486,539; 4,563,419; and 4,868,104, the disclosures of each of which are incorporated herein by reference in their entireties).

The antibodies of this invention can be used in in vitro, in vivo and/or in in situ assays to detect a biomarker protein or peptide of this invention.

Also as used herein, the terms peptide and polypeptide are used to describe a chain of amino acids, which correspond to those encoded by a nucleic acid. A peptide usually describes a chain of amino acids of from two to about 30 amino acids and polypeptide usually describes a chain of amino acids having more than about 30 amino acids. The term polypeptide can refer to a linear chain of amino acids or it can refer to a chain of amino acids, which have been processed and folded into a functional protein. It is understood, however, that 30 is an arbitrary number with regard to distinguishing peptides and polypeptides and the terms may be used interchangeably for a chain of amino acids around 30. The peptides and polypeptides of the present invention can be obtained by isolation and purification of the peptides and polypeptides from cells where they are produced naturally or by expression of a recombinant and/or synthetic nucleic acid encoding the peptide or polypeptide. The peptides and polypeptides of this invention can be obtained by chemical synthesis, by proteolytic cleavage of a polypeptide and/or by synthesis from nucleic acid encoding the peptide or polypeptide.

It is also understood that the peptides and polypeptides of this invention may also contain conservative substitutions where a naturally occurring amino acid is replaced by one having similar properties and which does not alter the function of the peptide or polypeptide. Such conservative substitutions are well known in the art. Thus, it is understood that, where desired, modifications and changes can be made in the nucleic acid and/or amino acid sequence of the peptides and polypeptides of the present invention and still obtain a peptide or polypeptide having like or otherwise desirable characteristics. Such changes can occur in natural isolates or can be synthetically introduced using site-specific mutagenesis, the procedures for which, such as mis-match polymerase chain reaction (PCR), are well known in the art. One of skill in the art will also understand that polypeptides and nucleic acids that contain modified amino acids and nucleotides, respectively (e.g., to increase the half-life and/or the therapeutic efficacy of the molecule), can be used in the methods of the invention.

“Nucleic acid” as used herein refers to single- or double-stranded molecules which may be DNA, comprised of the nucleotide bases A, T, C and G, or RNA, comprised of the bases A, U (substitutes for T), C, and G. The nucleic acid may represent a coding strand or its complement. Nucleic acids may be identical in sequence to a sequence that is naturally occurring or may include alternative codons that encode the same amino acid as that which is found in the naturally occurring sequence. Furthermore, nucleic acids may include codons that represent conservative substitutions of amino acids as are well known in the art. The nucleic acids of this invention can also comprise any nucleotide analogs and/or derivatives as are well known in the art.

As used herein, the term “isolated nucleic acid” means a nucleic acid separated or substantially free from at least some of the other components of the naturally occurring organism, for example, the cell structural components commonly found associated with nucleic acids in a cellular environment and/or other nucleic acids. The isolation of nucleic acids can therefore be accomplished by well-known techniques such as cell lysis followed by phenol plus chloroform extraction, followed by ethanol precipitation of the nucleic acids. The nucleic acids of this invention can be isolated from cells according to methods well known in the art for isolating nucleic acids. Alternatively, the nucleic acids of the present invention can be synthesized according to standard protocols well described in the literature for synthesizing nucleic acids. Modifications to the nucleic acids of the invention are also contemplated, provided that the essential structure and function of the peptide or polypeptide encoded by the nucleic acid are maintained.

The present invention further provides a kit for detection and/or quantification of the biomarkers of this invention. In some embodiments, Such a kit can comprise one or more antibodies of this invention and/or one or more aptamers of this invention, along with suitable buffers, wash solutions, dilution buffers, secondary antibodies, detection reagents, etc. for the detection of antigen/antibody complex formation and/or aptamer/target formation under various conditions. In another embodiment, a kit of this invention can comprise a nucleic acid probe or primer that is complementary to a nucleotide sequence encoding a biomarker of this invention, along with suitable buffers, wash solutions, dilution buffers, detection reagents, etc. for the detection of nucleic acid hybridization under various conditions.

Thus, in some embodiments, the present invention provides a kit comprising an antibody that specifically reacts with a biomarker associated with physical activity and reagents for detecting antigen/antibody complex formation.

Further provided is a kit comprising an aptamer that specifically reacts with a biomarker associated with physical activity and reagents for detecting aptamer/target molecule complex formation.

In addition, a kit is provided herein, comprising a nucleic acid that hybridizes under high stringency conditions with a nucleic acid encoding a biomarker associated with physical activity and reagents for detecting nucleic acid hybridization complex formation.

The present invention is more particularly described in the Examples set forth below, which are not intended to be limiting of the embodiments of this invention.

### EXAMPLES

### Summary of Studies

The present invention is directed to the detection and/or measurement of biomarkers associated with a change in physical performance in mammals. The goal of the studies described herein was to determine if a biomarker could be found which would accurately predict physical performance decrement. State-of-the-art measurements of oxygen utilization and carbon dioxide production were made during an 8-hour period of continuous exercise. The effects of a carbohydrate supplement were evaluated using a crossover design. Various performance measures were also made during the 8-hour period. Saliva samples were also collected prior to the start of work and at various times. Mass spectrometry was used to evaluate the chemical composition of the samples. Comparisons were made between samples obtained prior to exercise and at 4 hours. An automated software tool was used to identify biomarkers. In one study, a biomarker of molecular weight approximately 1,365 Daltons was identified. Statistical analysis of data shows that measurement of this biomarker is as useful as state-of-the-art gas measurements with regard to detecting changes in physical performance and detecting the effect of carbohydrate supplementation on physical performance.

### Laboratory Physical Performance Study

Nine healthy subjects were recruited at the University of Montana (Missoula, Mont.) for participation in the study. Subjects were required to fast for 12 hours prior to initiation of the study at 6 am. Weight, height and demographic information were collected. Total body fat was estimated using whole body immersion. During each 1 hour segment of the study participants were required to: 1) pedal a stationary bicycle for 25 minutes, 2) walk on an inclined treadmill for 25 minutes, 3) rest for 5 minutes. A 5 minute transition period was also included for movement from the treadmill to the stationary bicycle. Participants were required to meet specific physical work goals that were described on computer screens at the work stations. The work regimen was varied during each 25 minute period and averaged 70% of maximal work output. The pattern was repeated every hour for 8 hours. Saliva samples were collected by spitting into a 50 ml conical centrifuge tube with protease inhibitor cocktail added, during the 5 minute rest period. These samples were collected just prior to the start of the study (0 hour) and at the end of the 4, 5, 6, 7 and 8 hour work intervals. Blood samples were collected prior to start, and at 4 and 8 hours. A muscle biopsy was taken from the thigh muscle prior to the start of the study and at 8 hours. and 4 perform an a perceived Saliva samples were acquired from nine human subjects, at various times, during an 8 hour period of intense physical exercise consisting of pedaling a stationary bicycle and walking on an inclined treadmill. During the period utilization of oxygen (VO₂, liters/minute) and production of CO₂ (VCO₂, liters/minute) was measured. Average revolutions per minute (RPM) on the stationary cycle and a self-reported assessment of the difficulty in performing the required physical activity were also provided (RPE). In the case of RPE higher scores indicate greater difficulty and lower scores less. An estimate of the rate of carbohydrate oxidation (CHO, grams/min) was calculated using the formula, CHO=(4.55*VCO₂)−(3.21*VO₂). The rate of fat consumption (FAT, grams/minute) was calculated using, FAT=1.67*(VO₂—VCO₂).

The study was repeated twice in a crossover design where subjects received in one instance, Trial A, a carbohydrate supplement or a placebo supplement, Trial B, at 4 hours. Subjects were randomized to each trial so that trial A was conducted first in some subjects and last in others. At least two weeks passed between trial periods to reduce any carryover effect. All samples were stored at −80° C. prior to evaluation.

### Discovery of Saliva Biomarker

Saliva samples were initially depleted of anionic proteins and peptides by addition of positively charged beads made for this purpose (Brukker). A typical Matrix Assisted Laser Desorption/Ionization (MALDI) analysis was then performed. Briefly, the depleted saliva was mixed with cinapinic acid and spotted onto a gold plate. The plate was then interrogated with a laser to cause desorption and ionization of proteins and peptides. Spectra were then collected in the range of 1-3 kDa.

Spectra were compiled into groups based on the time at which the samples were obtained and which trial was used. An automated biomarker discovery tool, ClinProt® (Bruuker) was used to find biomarkers. The software finds peaks that are differ between treatment groups, in this case time and trial. These peaks heights are then used to determine if a correlation exists between the biomarker response and changes in physical performance of subjects.

### Baseline Measurements and Demographics of the Study Group

Table 1 shows these data. The study population was recruited from students and faculty at the University of Montana. Many of the participants are amateur athletes and were highly motivated to participate in the study. The study population has an average age of 28 years, 39.1 CV %, and an average body weight of 75 kg. The largest observed variation occurs in estimated body fat index, average 8.9%, CV % 60.1 while surprisingly little variation occurs in weight normalized VO2 Max, 67.6 ml/kg-min, 12.4 CV %. These parameters support the notion that participants are relatively young and fit.

### Comparison of the Biomarker to Other Measures of Performance

The biomarkers of this invention should prove useful in determining the physical fitness of an individual during prolonged exercise. If the biomarker is useful in this regard then levels of the biomarker would be different depending on the time at which they were measured and if carbohydrate supplementation was given. Table 3 shows p-values for ANOVA terms for the various measured parameters. The table shows that the biomarker is as good as either CHO or FAT as a measure of treatment effects and change occurring over time.

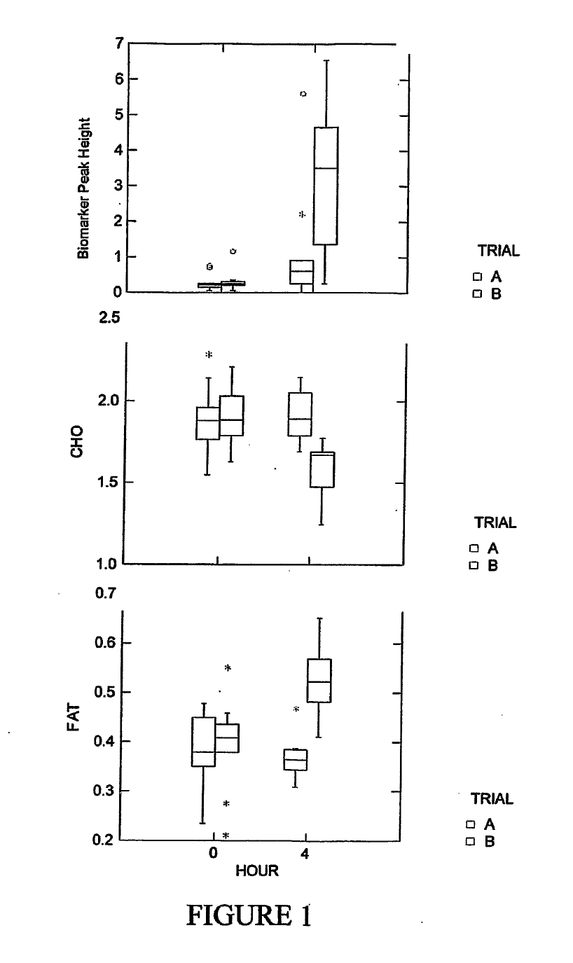

FIG. 1 shows that the median biomarker peak height increases by approximately 3 fold in trail B (placebo) treated subjects and more than 30 fold in subjects that receive the carbohydrate supplement. CHO shows a pattern of change that is the inverse of the biomarker change whereas FAT shows changes is mirrors change in the biomarker.

The data presented here show that the predictive power of the biomarker is similar to the power achieved by measuring CHO or FAT. The immediate utility of this approach is that the biomarker can be used instead of measuring exhaled gases. The measurement of exhaled gases requires large and expensive devices. Moreover, gas measurements cannot be adapted to assessment of energy utilization outside the laboratory. Measurement of the biomarker using a disposable and cheap dipstick technology enables the evaluation of the effect of interventions to increase human performance or to increase amounts of available carbohydrate stores available for the performance of work.

### Identification and Characterization of Biomarkers

Five potential performance biomarker peptides previously identified by statistical analysis of Matrix Assisted Laser Desorption Ionization Time of Flight Mass Spectrometry (MALDI-TOF) data were further analyzed by Electro-Spray Ionization Quadrupole Ion-Trap Mass Spectrometry (ESI-MS). The purpose of this analysis was to provide positive sequence identification of the peptides. The identification was performed by direct injection of pre-fractionated peptides from human saliva. The spectral data was submitted to an on-line search engine (MASCOT).

The identifications in Table 5 were made by the search engine. The peptide m/z is the mass to charge ratio of the parent peptide fragmented in the ESI-MS in order to provide the spectral data for the search. The identified protein is the potential parent protein that the peptide originated from and the sequence is the best fit amino-acid sequence for the spectral data. The ion score is a statistical parameter giving the quality of the fit.

### Biomarker Protein Clips

Fresh human saliva was collected and immediately fractionated by spin-filtration through a 10 kDa MWCO filter. The <10 kDa fraction was de-salted over a 4.6 mm×50 mm Agilent mRP Hi-recovery protein column. The peptides were eluted with ˜1 ml 70% acetonitrile and evaporated to dryness. The dried peptides were reconstituted in 100 μl of 50% methanol with 0.1% formic acid. The solution was analyzed by direct infusion into a Finnigan LCQ-DUO ESI ion-trap mass spectrometer equipped with an electrospray source. Tandem mass spectrometric analysis was performed on several observed peptide ions. Peptides were isolated with a width of 5 m/z and fragmented with sufficient collision energy to reduce the parent intensity to less than 10% of the total signal. CID spectra were averaged for each analyzed peptide and the data were converted to MASCOT generic data files. The files were submitted to MASCOT's MS/MS ion search algorithm with no enzyme restriction and no protein mass specified.

Several peptide ions and their resulting MS/MS fragments had high scores for matching human salivary proteins. Several other peptides had high scores for matching and good sequence homology of other human proteins not expected in saliva (Table 5).

Although the present invention has been described with reference to specific details of certain embodiments thereof, it is not intended that such details should be regarded as limitations upon the scope of the invention except as and to the extent that they are included in the accompanying claims.

Throughout this application, various patents, patent publications and non-patent publications are referenced. The disclosures of these patents, patent publications and non-patent publications in their entireties are incorporated by reference into this application in order to more fully describe the state of the art to which this invention pertains.

TABLE 1
Baseline Measurements and Demographics of the Study Group.

| | | | | Lean | | |
|---|---|---|---|---|---|---|
| | | | | Body | Cycle | Treadmill |
| | Height | Weight | % Body | Mass | VO2max | VO2max |
| Age | (cm) | (kg) | Fat | (kg) | (ml/kg/min) | (ml/kg/min) |
| 48 | 175.3 | 84.3 | 17.8 | 69.3 | 42.96 | 52.27 |
| 19 | 177.8 | 62.4 | 3.1 | 60.5 | 67.63 | 70.44 |
| 21 | 182.9 | 79.5 | 4.8 | 75.7 | 58.39 | 66.02 |
| 23 | 175.3 | 67.3 | 4.5 | 64.3 | 54.63 | 62.65 |
| 28 | 182.9 | 89.4 | 14.0 | 76.9 | 55.64 | 59.18 |
| 22 | 180.3 | 84.1 | 7.6 | 77.7 | 59.58 | 60.94 |
| 25 | 177.8 | 64.7 | 9.0 | 58.9 | 55.31 | 56.98 |
| 46 | 182.9 | 84.0 | 14.7 | 71.6 | 58.67 | 57.62 |
| 21 | 167.6 | 60.0 | 4.5 | 57.3 | 65.8 | 74.03 |

TABLE 2
Raw data from study.

| | | | Biomarker | | | | | | |
|---|---|---|---|---|---|---|---|---|---|
| | | | Peak | | | | | | |
| Subject | HOUR | TRIAL | Height | CHO | FAT | VO2 | VCO2 | RPM | RPE |
| 1 | 4 | A | 0 | 2.07 | 0.37 | 2.31 | 2.08 | | 12 |
| 2 | 4 | A | 0.1 | 1.92 | 0.47 | 2.38 | 2.10 | 86 | 13 |
| 3 | 4 | A | 0.3 | 2.03 | 0.38 | 2.29 | 2.06 | | 11 |
| 4 | 4 | A | 0.25 | 1.69 | 0.39 | 2.05 | 1.82 | 63 | 11 |
| 5 | 4 | A | 0.9 | 2.14 | 0.35 | 2.32 | 2.11 | 83 | 11 |
| 6 | 4 | A | 2.2 | 1.87 | 0.33 | 2.07 | 1.87 | 85 | 12 |
| 7 | 4 | A | 5.6 | 1.73 | 0.35 | 2.01 | 1.80 | 88 | 11 |
| 8 | 4 | A | 0.6 | | | | | 87 | 10 |
| 9 | 4 | A | 0.6 | 1.84 | 0.31 | 2.00 | 1.82 | 86 | 11 |
| 1 | 0 | A | 0.15 | 2.14 | 0.38 | 2.37 | 2.14 | | 10 |
| 2 | 0 | A | 0.75 | 1.86 | 0.48 | 2.36 | 2.07 | 86 | 11 |
| 3 | 0 | A | 0.05 | 2.28 | 0.23 | 2.18 | 2.04 | 73 | 9 |
| 4 | 0 | A | 0.15 | 1.55 | 0.45 | 2.07 | 1.80 | 66 | 11 |
| 5 | 0 | A | 0.7 | 1.96 | 0.45 | 2.38 | 2.11 | 78 | 11 |
| 6 | 0 | A | 0.25 | 1.88 | 0.34 | 2.10 | 1.90 | 84 | 12 |
| 7 | 0 | A | 0.2 | 1.74 | 0.35 | 2.01 | 1.80 | 83 | 11 |
| 8 | 0 | A | 0.15 | 1.89 | 0.45 | 2.31 | 2.05 | 88 | 10 |
| 9 | 0 | A | 0.2 | 1.76 | 0.37 | 2.07 | 1.85 | 91 | 11 |
| 1 | 4 | B | 3.5 | 1.48 | 0.58 | 2.28 | 1.94 | | 12 |
| 2 | 4 | B | 5.1 | 1.77 | 0.52 | 2.39 | 2.07 | 100 | 13 |
| 3 | 4 | B | 1.35 | 1.68 | 0.54 | 2.34 | 2.02 | 71 | 12 |
| 4 | 4 | B | 3.6 | 1.47 | 0.41 | 1.94 | 1.69 | 70 | 11 |
| 5 | 4 | B | 0.4 | 1.69 | 0.52 | 2.31 | 2.00 | 77 | 12 |
| 6 | 4 | B | 2.7 | 1.74 | 0.41 | 2.14 | 1.89 | 86 | 12 |
| 7 | 4 | B | 4.65 | 1.24 | 0.57 | 2.08 | 1.74 | 92 | 11 |
| 8 | 4 | B | 0.25 | 1.26 | 0.65 | 2.27 | 1.88 | 84 | 10 |
| 9 | 4 | B | 6.55 | 1.67 | 0.48 | 2.23 | 1.94 | 100 | 11 |
| 1 | 0 | B | 0.25 | 1.88 | 0.46 | 2.34 | 2.06 | | 12 |
| 2 | 0 | B | 0.35 | 2.15 | 0.38 | 2.37 | 2.14 | 96 | 12 |
| 3 | 0 | B | 0.3 | 2.20 | 0.28 | 2.20 | 2.04 | 74 | 11 |
| 4 | 0 | B | 1.15 | 2.03 | 0.21 | 1.94 | 1.82 | 68 | 11 |
| 5 | 0 | B | 0.2 | 1.92 | 0.44 | 2.32 | 2.06 | 68 | 12 |
| 6 | 0 | B | 0.15 | 1.79 | 0.41 | 2.16 | 1.92 | 81 | 12 |
| 7 | 0 | B | 0.05 | 1.63 | 0.42 | 2.07 | 1.82 | 88 | 10 |
| 8 | 0 | B | 0.25 | 1.63 | 0.55 | 2.33 | 2.00 | 87 | 9 |
| 9 | 0 | B | 0.25 | 1.87 | 0.38 | 2.18 | 1.95 | 98 | 11 |

TABLE 3
p-values from ANOVA of data. Hour and Trial are single effects
and Hour*Trial is the interaction term.

| | Hour | Trial | Hour*Trial |
|---|---|---|---|
| Biomarker | 0.0000 (F = 15) | 0.043 | 0.051 |
| CHO | 0.020 | 0.014 | 0.011 |
| FAT | 0.046 | 0.007 | 0.009 |
| VO₂ | 0.839 | 0.635 | 0.747 |
| CO₂ | 0.330 | 0.626 | 0.526 |
| RPM | 0.601 | 0.614 | 0.889 |
| RPE | 0.080 | 0.285 | 0.720 |

TABLE 4
New biomarkers identified

Copper fractionation bead chemistry, samples not present in time = 0,
before exercise begins, but in subsequent times, starting at 4 hours when
either supplement or placebo is administered, during trial A without
carbohydrate supplementation (all in Daltons where it is assumed that
charge state is +1):
1323, 2637, 3048, 3208, 1728, 3049, 3204, 4396, 4071, 4396, 4055,
4938, 4929, 4066, 4844, 5228
Copper chemistry, trial with carbohydrate, trial B:
1573, 1730, 1442, 1729, 3048, 4961, 4403, 4388, 4402, 4961, 4979
Copper chemistry where a LOSS occurs during exercise, trial A:
1234, 1478
Copper chemistry where a LOSS occurs during exercise, trial B:
2340, 5521, 4979
Weak cation exchange chemistry as above, trial A showed nothing, trial B:
2935, 1880, 2934, 2510
Weak cation where LOSS occurred relative to time = 0:
1744, 1328

TABLE 5
Identified proteins

| Peptide m/z | Identified Protein | Sequence | Ion Score |
|---|---|---|---|
| 874.0 | Human Proline Rich Protein 12 | QGPPPPGKP (SEQ ID NO:1) | 45 |
| 971.0 | Human Basic PRP P-E | GPPPPPGKPQ (SEQ ID NO:2) | 28 |
| 1226.0 | TNF alpha-inducing factor | RGNRSPVPDVE (SEQ ID NO:3) | 14 |
| 1262.0 | MTHF Reductase | VLYLSGEPNRN (SEQ ID NO:4) | 17 |

TABLE 6

| | | | | | |
|---|---|---|---|---|---|
| Salivary peptides sequenced. | | | | | |
| Source | | | | | |
| (hours) | Species | Query | Observed | Mr (calc) | Peptide Discovered |

| | | | | | |
|---|---|---|---|---|---|
| 0 | Human | 1 | 1200.6400 | 2400.2648 | HAVEEVVKEVVGHAKETGEKAI |
| | | | | | (SEQ ID NO:5) |
| 0 | All | 1 | 1200.6400 | 1199.6339 | GPGIFPPPPPQP |
| | | | | | (SEQ ID NO:6) |
| 0 | Human | 1 | 1222.7300 | 1221.7121 | PLLFLPPAAQVG |
| | | | | | (SEQ ID NO:7) |
| 0 | All | 1 | 1222.7300 | 1222.6710 | AKLTTPPFLGH |
| | | | | | (SEQ ID NO:8) |
| 0 | Human | 1 | 1318.6400 | 1317.6757 | GPGFVPPPPPPPY |
| | | | | | (SEQ ID NO:9) |
| 0 | All | 1 | 1318.6400 | 1317.6757 | GPGFVPPPPPPPY |
| | | | | | (SEQ ID NO:10) |
| 0 | Human | 1 | 578.3600 | 1156.6451 | SPALLGVQKGTS |
| | | | | | (SEQ ID NO:11) |
| 0 | All | 1 | 578.3600 | 1730.9090 | KFTEAGVPIIGDDIKS |
| | | | | | (SEQ ID NO:12) |
| 0 | Human | 1 | 658.3600 | 1314.7084 | GPGRTPPPPPAPY |
| | | | | | (SEQ ID NO:13) |
| 0 | All | 1 | 658.3600 | 1314.7408 | HKTNTRVLSLF |
| | | | | | (SEQ ID NO:14) |
| 0 | Human | 1 | 764.4500 | 762.3912 | NVSSPFI |
| | | | | | (SEQ ID NO:(SEQ ID NO:15) |
| 0 | All | 1 | 764.4500 | 762.3946 | LTSSMKP |
| | | | | | (SEQ ID NO:16) |
| 0 | Human | 1 | 963.0900 | 2884.5109 | LNQEGKFLYISETVSIYLGLSQVEL |
| | | | | | (SEQ ID NO:17) |
| 0 | All | 1 | 963.0900 | 963.4524 | CPPPPPPYP |
| | | | | | (SEQ ID NO:18) |
| 4 | Human | 1 | 1057.0900 | 3166.6961 | ITCRASQDIKNFLAWLQQKPGKAPKSL |
| | | | | | (SEQ ID NO:19) |
| 4 | Human | 1 | 1057.0900 | 3166.6961 | ITCRASQDIKNFLAWLQQKPGKAPKSL |
| | | | | | (SEQ ID NO:20) |
| 4 | All | 1 | 1057.0900 | 3168.6050 | VERSLGIYSGDPISPDIESGLHGRSGVTSLA |
| | | | | | (SEQ ID NO:21) |
| 4 | Human | 1 | 1168.2700 | 1167.6724 | HRVKILTNSTL |
| | | | | | (SEQ ID NO:22) |
| 4 | All | 1 | 1168.2700 | 3502.8380 | AKVVNIGGLGVGFDSAKPLTGEFKKISETGKGM |
| | | | | | (SEQ ID NO:23) |
| 4 | Human | 1 | 1454.6400 | 1452.6844 | APVGTGTAPGGGPGSGVD |
| | | | | | (SEQ ID NO:24) |
| 4 | All | 1 | 1454.6400 | 4359.2022 | VASTALRQPNGTRFSPSGKILYVTDTGAASGSLSVPFDEISY |
| | | | | | (SEQ ID NO:25) |
| 4 | Human | 1 | 841.1800 | 2519.1975 | SACLAKHQDGLWHAARITDVDNG |
| | | | | | (SEQ ID NO:26) |
| 4 | All | 1 | 841.1800 | 1678.7937 | EPITVTVDVKADYDD |
| | | | | | (SEQ ID NO:27) |
| 4 | Human | 1 | 977.3600 | 977.4162 | RRSDGEDGS |
| | | | | | (SEQ ID NO:28) |
| 4 | All | 1 | 977.3600 | 1952.9843 | VPAAPPPAAGDDQLPAEAPVA |
| | | | | | (SEQ ID NO:29) |
| 8 | Human | 1 | 1794.4500 | 5379.5035 | NAKNSLYLQMNSLRDEDTAVYYCARCARGEAGVRGSYYRAFDIWGQG |
| | | | | | (SEQ ID NO:30) |
| 8 | All | 1 | 1794.4500 | 5380.6869 | RKDSSRSPERYRNRNQISKGIEYSRSNSISPDRISSNSSRRYPNP |
| | | | | | (SEQ ID NO:31) |
| 8 | All | 1 | 1794.4500 | 5379.5035 | NAKNSLYLQMNSLRDEDTAVYYCARCARGEAGVRGSYYRAFDIWGQG |
| | | | | | (SEQ ID NO:32) |
| 8 | All | 1 | 1794.4500 | 5378.7298 | HWVRQAPGKGLEWVAVISPDGSMKYYADSVKGRFISRDNSKKTVF |
| | | | | | (SEQ ID NO:33) |
| 8 | Human | 1 | 611.9100 | 1221.6142 | PARLKGNFDY |
| | | | | | (SEQ ID NO:34) |
| 8 | Human | 1 | 611.9100 | 1221.6142 | PARLKGNFDY |
| | | | | | (SEQ ID NO:35) |
| 8 | All | 1 | 611.9100 | 1221.7056 | IPLIRWKCPP |
| | | | | | (SEQ ID NO:36) |

Source of sample was saliva pooled from subjects that are source for other data in the present application. Source saliva was pooled from three subjects and then analyzed. Time is hours after start of trial arm where no carbohydrate was administered. Species represents best statistical match with MASCOT database (Electrophoresis, 20(18) 3551-67 (1999)) within criteria. In other words, searches for sequences were restricted in some cases to human only. In the case of “All,” all spectra in
MASCOT were examined for similarity. Query links to the MASCOT search and can be followed to NCBI/BLAST for a more complete description. Observed refers to the observed m/Z of parent ion species. Mr (calc) refers to the calculated molecular weight of the entire peptide. A charge ionization of +1, +2 and +3 ionization, and variable acetylation was considered during database search.

1. A method of detecting an improvement in the performance of a physical or athletic activity in a subject upon administration to the subject of a performance enhancing material and/or upon contact of the subject with a performance enhancing material and/or upon implementation of a performance enhancing activity by the subject, comprising:
a) measuring an amount of a biomarker in a first sample of a test subject prior to performance of the physical or athletic activity;
b) measuring the amount of a biomarker in a first sample of a control subject prior to performance of the physical or athletic activity;
c) administering to the subject the performance enhancing material and/or contacting the subject with the performance enhancing material and/or implementing the performance enhancing activity to the test subject;
d) having the test subject and control subject perform the physical or athletic activity;
e) measuring the amount of the biomarker in a second sample of the test subject and measuring the amount of the biomarker in a second sample of the control subject after step (d);
f) comparing the amount of the biomarker in the first sample and the second sample of the test subject and comparing the amount of the biomarker in the first sample and the second sample of the control subject; and
g) comparing the change in the amount of the biomarker of step (e) in the test subject with the change in the amount of the biomarker of step (e) in the control subject, whereby an increase in the amount of biomarker in the test subject that is greater than an increase in the amount of the biomarker in the control subject detects an improvement in the performance of the physical or athletic activity in the subject upon administration to the subject of the performance enhancing material and/or contacting the subject with the performance enhancing material and/or upon implementation of a performance enhancing activity by the subject.

2. A method of detecting a change in the chemical energy source, (e.g., fat and/or carbohydrate) used by the body of a subject during performance of physical or athletic activity, upon administration to the subject of a performance enhancing material and/or upon contact of the subject with a performance enhancing material and/or upon implementation of a performance enhancing activity by the subject, comprising:
a) measuring an amount of a biomarker in a first sample of a test subject prior to performance of the physical or athletic activity;
b) measuring the amount of a biomarker in a first sample of a control subject prior to performance of the physical or athletic activity;
c) administering to the subject the performance enhancing material and/or contacting the subject with the performance enhancing material and/or implementing the performance enhancing activity to the test subject;
d) having the test subject and control subject perform the physical or athletic activity;
e) measuring the amount of the biomarker in a second sample of the test subject and measuring the amount of the biomarker in a second sample of the control subject after step (d);
f) comparing the amount of the biomarker in the first sample and the second sample of the test subject and comparing the amount of the biomarker in the first sample and the second sample of the control subject; and
g) comparing the change in the amount of the biomarker of step (e) in the test subject with the change in the amount of the biomarker of step (e) in the control subject, whereby an increase in the amount of biomarker in the test subject that is greater than or less than an increase in the amount of the biomarker in the control subject detects a change in the chemical energy source, (e.g., fat and/or carbohydrate) used by the body of the subject during performance of physical or athletic activity, upon administration to the subject of a performance enhancing material and/or upon contact of the subject with a performance enhancing material and/or upon implementation of a performance enhancing activity by the subject.

3. (canceled)

4. A method of detecting a decrement in the performance of a physical or athletic activity in a subject upon administration to the subject of a performance enhancing material and/or upon contact of the subject with a performance enhancing material and/or upon implementation of a performance enhancing activity by the subject, comprising:
a) measuring an amount of a biomarker in a first sample of a test subject prior to performance of the physical or athletic activity;
b) measuring the amount of a biomarker in a first sample of a control subject prior to performance of the physical or athletic activity;
c) administering to the subject the performance enhancing material and/or contacting the subject with the performance enhancing material and/or implementing the performance enhancing activity to the test subject;
d) having the test subject and control subject perform the physical or athletic activity;
e) measuring the amount of the biomarker in a second sample of the test subject and measuring the amount of the biomarker in a second sample of the control subject after step (d);
f) comparing the amount of the biomarker in the first sample and the second sample of the test subject and comparing the amount of the biomarker in the first sample and the second sample of the control subject; and
g) comparing the change in the amount of the biomarker of step (e) in the test subject with the change in the amount of the biomarker of step (e) in the control subject, whereby an increase in the amount of biomarker in the test subject that is less than an increase in the amount of the biomarker in the control subject detects a decrement in the performance of the physical or athletic activity in the subject upon administration to the subject of the performance enhancing material and/or contacting the subject with the performance enhancing material and/or upon implementation of a performance enhancing activity by the subject.

5. (canceled)

6. The method of claim 1, wherein the performance enhancing material is a dietary or nutritional supplement or food that consists primarily of sugars and carbohydrates; a nutritional supplement that contains fenugreek or fenugreek extracts; a nutritional supplement that is composed in part of antioxidants including vitamins C and E; green tea extract; an extract of the Aloe vera plant; melatonin; essential, non-essential and chemically modified amino acids; selenium; cobalt; magnesium; 1-alpha-carnitine; amphetamine; modafinil; creatinine; a dietary supplement composed principally of proteins including whey protein and yeast protein; anabolic steroids; erythropoietin; ephedrine; ginseng; caffeine; forskohlin; glutamine, arginine, whey protein; soy protein; egg albumin; casein; (nutritional compound described in U.S. Pat. No. 6,051,236 as a dry powder comprising protein, amino acids, CHO, vitamins C and E); growth hormone; vitamins, minerals; essential fatty acids; tricarboxylic acid cycle intermediates; free fatty acids; pyruvate; creatine; ipriflavone; pentose (e.g., a monosaccharide such as ribose, D-ribose, ribulose, xylitol, xylulose, any five carbon precursor of ribose; and/or cortisol blockers (e.g., phosphatidylserine, HMB, DHEA, CLA, anabolic steroids, creatine monohydrates, pregnenalone, Ipriflavone, super physiological levels of leucine, anabolic steroids, antioxidants, leucine metabolites, glutamic acid and its metabolites, glutamine and androstenedione).

7. The method of any of claim 1, wherein the performance enhancing activity is an exercise regimen that incorporates strength training and/or aerobic exercise; massage therapy; meditation; biofeedback; and/or hypnosis.

8. The method of claim 1, wherein the performance enhancing material is a device that emit photons in the near infra-red spectrum to increase energy output in muscles; clothing, including pants, shorts, socks, briefs, underwear, footwear, orthotics, ankle, knee and leg supports, orthotics and/or other devices designed to increase physical performance and/or reduce fatigue; and/or clothing, wraps, supports and/or other devices that cool muscles.

9. The method of any of claim 1, wherein the biomarker is a 1365 Dalton protein described herein, interleukin-6, interleukin-1 beta, G-CSF, interferon-gamma, interleukin-8, interleukin-9, MCP-1, MIP-beta, and/or TNF alpha

10. The method of any of claim 1, wherein an endogenously produced protein, glycoprotein or polymer, is measured as an standard useful to normalize concentrations of biomarkers measured (this is essentially an internal standard that can be used to correct for relative dilution of the saliva which is in turn dependent on the hydration of the subject and other factors including drugs and individual physiological characteristics).

11-28. (canceled)

29. The method of any of claim 1, wherein the biomarker is a peptide comprising the amino acid sequence of any of SEQ ID NOs:1-36, singly or in any combination.

30. The kit of any of claim 1, wherein the biomarker is a peptide comprising the amino acid sequence of any of SEQ ID NOs:1-36, singly or in any combination.


SEQUENCE LISTING (36 sequences)

SEQ ID NO: 1 - protein, 9 aa, Homo sapiens
QGPPPPGKP

SEQ ID NO: 2 - protein, 10 aa, Homo sapiens
GPPPPPGKPQ

SEQ ID NO: 3 - protein, 11 aa, Homo sapiens
RGNRSPVPDVE

SEQ ID NO: 4 - protein, 11 aa, Homo sapiens
VLYLSGEPNRN

SEQ ID NO: 5 - protein, 22 aa, Homo sapiens
HAVEEVVKEVVGHAKETGEKAI

SEQ ID NO: 6 - protein, 12 aa, Unknown
  MASCOT identified peptide sequence
GPGIFPPPPPQP

SEQ ID NO: 7 - protein, 12 aa, Homo sapiens
PLLFLPPAAQVG

SEQ ID NO: 8 - protein, 11 aa, Unknown
  MASCOT identified peptide sequence
AKLTTPPFLGH

SEQ ID NO: 9 - protein, 13 aa, Homo sapiens
GPGFVPPPPPPPY

SEQ ID NO: 10 - protein, 13 aa, Unknown
  MASCOT identified peptide sequence
GPGFVPPPPPPPY

SEQ ID NO: 11 - protein, 12 aa, Homo sapiens
SPALLGVQKGTS

SEQ ID NO: 12 - protein, 16 aa, Unknown
  MASCOT identified peptide sequence
KFTEAGVPIIGDDIKS

SEQ ID NO: 13 - protein, 13 aa, Homo sapiens
GPGRIPPPPPAPY

SEQ ID NO: 14 - protein, 11 aa, Unknown
  MASCOT identified peptide sequence
HKTNTRVLSLF

SEQ ID NO: 15 - protein, 7 aa, Homo sapiens
NVSSPFI

SEQ ID NO: 16 - protein, 7 aa, Unknown
  MASCOT identified peptide sequence
LTSSMKP

SEQ ID NO: 17 - protein, 25 aa, Homo sapiens
LNQEGKFLYISETVSIYLGLSQVEL

SEQ ID NO: 18 - protein, 9 aa, Unknown
  MASCOT identified peptide sequence
CPPPPPPYP

SEQ ID NO: 19 - protein, 27 aa, Homo sapiens
ITCRASQDIKNFLAWLQQKPGKAPKSL

SEQ ID NO: 20 - protein, 27 aa, Homo sapiens
ITCRASQDIKNFLAWLQQKPGKAPKSL

SEQ ID NO: 21 - protein, 31 aa, Unknown
  MASCOT identified peptide sequence
VERSLGIYSGDPISPDIESGLHGRSGVTSLA

SEQ ID NO: 22 - protein, 10 aa, Homo sapiens
HRVKLTNSTL

SEQ ID NO: 23 - protein, 33 aa, Unknown
  MASCOT identified peptide sequence
AKVVNIGGLGVGFDSAKPLTGEFKKISETGKGM

SEQ ID NO: 24 - protein, 18 aa, Homo sapiens
APVGTGTAPGGGPGSGVD

SEQ ID NO: 25 - protein, 42 aa, Unknown
  MASCOT identified peptide sequence
VASTALRQPNGTRFSPSGKILYVTDTGAASGSLSVPFDEISY

SEQ ID NO: 26 - protein, 23 aa, Homo sapiens
SACLAKHQDGLWHAARITDVDNG

SEQ ID NO: 27 - protein, 15 aa, Unknown
  MASCOT identified peptide sequence
EPITVTVDVKADYDD

SEQ ID NO: 28 - protein, 9 aa, Homo sapiens
RRSDGEDGS

SEQ ID NO: 29 - protein, 21 aa, Unknown
  MASCOT identified peptide sequence
VPAAPPPAAGDDQLPAEAPVA

SEQ ID NO: 30 - protein, 47 aa, Homo sapiens
NAKNSLYLQMNSLRDEDTAVYYCARCARGEAGVRGSYYRAFDIWGQG

SEQ ID NO: 31 - protein, 45 aa, Unknown
  MASCOT identified peptide sequence
RKDSSRSPERYRNRNQISKGIEYSRSNSISPDRISSNSSRRYPNP

SEQ ID NO: 32 - protein, 47 aa, Unknown; identical to SEQ ID NO: 30
  MASCOT identified peptide sequence

SEQ ID NO: 33 - protein, 46 aa, Unknown
  MASCOT identified peptide sequence
HWVRQAPGKGLEWVAVISPDGSMKYYADSVKGRFTISRDNSKKTVF

SEQ ID NO: 34 - protein, 10 aa, Homo sapiens
PARLKGNFDY

SEQ ID NO: 35 - protein, 10 aa, Unknown
  MASCOT identified peptide sequence
PARLKGNFDY

SEQ ID NO: 36 - protein, 10 aa, Homo sapiens
IPLIRWKCPP